(19) United States
(12) Patent Application Publication
(10) Pub. No.: US 2024/0290449 A1
(43) Pub. Date: Aug. 29, 2024

(54) REPORT GENERATION FOR RADIOLOGY

(71) Applicant: IX Innovation LLC, Seattle, WA (US)

(72) Inventors: Jeffrey Roh, Seattle, WA (US); Justin Esterberg, Mesa, AZ (US); Adam Benson, Missoula, MT (US); Julie Benson, Missoula, MT (US); John Cronin, Jericho, VT (US); Seth Cronin, Essex Junction, VT (US); Michael John Baker, Georgia, VT (US)

(73) Assignee: IX Innovation LLC, Seattle, WA (US)

(21) Appl. No.: 18/114,136

(22) Filed: Feb. 24, 2023

(51) Int. Cl. G16H 15/00; G16H 30/20; G16H 30/40

(52) U.S. Cl. CPC G16H 15/00; G16H 30/20; G16H 30/40; G06V 10/74

(57) ABSTRACT

Described herein are techniques for generating a radiological report. In some cases, some or all of the radiological report may be generated during a medical imaging to which the report relates, such that the report is generated contemporaneously with or in real time during the imaging. In some embodiments described herein, some or all of the radiological report may be generated automatically, without intervention from an imaging technician or from a radiologist. Such an automatic generation of radiological report content may be performed in some embodiments through comparison of information regarding a medical imaging of a patient to information regarding one or more prior medical imaging's of the patient or other patients. In response to detecting a match between information regarding a medical imaging of a patient and information regarding a prior medical imaging, information relating to the prior medical imaging may be used to generate the radiological report.

### FIELD

Some embodiments described herein relate to medical report generation, such as by comparing data for a current patient to data for one or more prior patients and, in response to detecting a match with a prior patient, generating a radiology report that includes content from a report for the prior patient.

### BACKGROUND

A medical examination of a patient may include performing a medical imaging of the patient. With medical imaging, a scan of the patient may be performed to generate imagery of the structure and/or function of the patient's anatomy at a time. A structural medical image may depict a patient's anatomy in a region under examination. A functional medical image may depict metabolic activity of a patient's anatomy in such a region under examination. Examples of medical imaging technologies include ultrasound, magnetic resonance imaging (MRI), computerized tomography (CT), and x-ray, among others.

After a medical image (or collection of images) is obtained by an imaging technician, such as minutes, hours, or even days later, the image(s) may be interpreted by a radiologist. The radiologist is a medical doctor who is different from the imaging technician and may not have been present during the medical imaging, and in some cases may not be in the same building or same geographic area as where the imaging was conducted. The radiologist's findings from analysis of the image may be set out in a radiology report. The report may also include the image(s) and information regarding the patient or the medical imaging.

### SUMMARY

In some embodiments, there is provided a method for medical report generation, which includes: comparing, by a device, data regarding a patient to prior patient data for one or more prior patients, the data regarding the patient includes information regarding a medical imaging of the patient and the prior patient data for each of the one or more prior patients includes information regarding a prior medical imaging of a prior patient, the comparing includes generating, via execution of an image recognition algorithm by the device, for each of the prior patient data regarding the one or more prior patients, a measure of a match between the data regarding the patient and prior patient data for a prior patient; determining, by the device, based at least in part on the measures of match between the data regarding the patient and the prior patient data for each prior patient of the one or more prior patients, there is a match between the data regarding the patient and prior patient data for a prior patient; generating, by the device, based on the determination of the match, a radiology report, the radiology report includes the medical imaging of the patient related to the comparison; and outputting, by the device, for display within a user interface (UI), the generated radiology report.

According to some embodiments, described herein is a system (e.g., an apparatus) for the advanced image stabilization. The apparatus includes at least one processor; and at least one storage medium having encoded thereon executable instructions that, when executed by the at least one processor, cause the at least one processor to carry out a method or methods similar to the methods discussed herein. According to some embodiments, such methods include: comparing, by a device, data regarding a patient to prior patient data for one or more prior patients, the data regarding the patient includes information regarding a medical imaging of the patient and the prior patient data for each of the one or more prior patients includes information regarding a prior medical imaging of a prior patient, the comparing includes generating, via execution of an image recognition algorithm by the device, for each of the prior patient data regarding the one or more prior patients, a measure of a match between the data regarding the patient and prior patient data for a prior patient; determining, by the device, based at least in part on the measures of match between the data regarding the patient and the prior patient data for each prior patient of the one or more prior patients, there is a match between the data regarding the patient and prior patient data for a prior patient; generating, by the device, based on the determination of the match, a radiology report, the radiology report includes the medical imaging of the patient related to the comparison; and outputting, by the device, for display within a user interface (UI), the generated radiology report.

According to some embodiments, described herein is at least one non-transitory computer-readable storage medium that has tangibly stored thereon, or tangibly encoded thereon, computer readable instructions that when executed by at least one processor of a device cause the at least one processor to perform a method for advanced image stabilization. The computer-executed method, as executed from the computer-executable instructions stored on the non-transitory computer-readable storage medium, includes: comparing, by a device, data regarding a patient to prior patient data for one or more prior patients, the data regarding the patient includes information regarding a medical imaging of the patient and the prior patient data for each of the one or more prior patients includes information regarding a prior medical imaging of a prior patient, the comparing includes generating, via execution of an image recognition algorithm by the device, for each of the prior patient data regarding the one or more prior patients, a measure of a match between the data regarding the patient and prior patient data for a prior patient; determining, by the device, based at least in part on the measures of match between the data regarding the patient and the prior patient data for each prior patient of the one or more prior patients, there is a match between the data regarding the patient and prior patient data for a prior patient; generating, by the device, based on the determination of the match, a radiology report, the radiology report includes the medical imaging of the patient related to the comparison; and outputting, by the device, for display within a user interface (UI), the generated radiology report.

According to some embodiments, the medical imaging of the patient includes information related to at least one of medical image data, sensor data and notes data related to a dictation of a clinician regarding the medical image data and/or the patient.

According to some embodiments, comparing the data regarding the patient to the prior patient data for the one or more prior patients includes, for a prior patient: determining one or more first items of information included in the data regarding the patient; determining one or more second items of information included in the prior patient data for the prior patient; and generating the measure of the match between the data regarding the patient and the prior patient data for the prior patient based on comparing the one or more first items of information and the one or more second items of information.

According to some embodiments, the one or more first items of information includes an indication and the medical image data for the medical imaging for the patient; the one or more second items of information includes notes of a clinician regarding the prior medical imaging of the prior patient; and comparing the one or more first items of information and the one or more second items of information includes comparing the indication and the medical image data for the patient to the notes of the clinician regarding the prior medical imaging of the prior patient.

According to some embodiments, generating the radiology report by including in the radiology report at least some content from the prior report includes populating the radiology report with information regarding an impression of a clinician for the prior patient and/or a prior procedure performed for the prior patient.

According to some embodiments, comparing the data regarding the patient to the prior patient data for the one or more prior patients includes analyzing the data regarding the patient and the prior patient data for the one or more prior patients using at least one trained model, the measure of the match being an output of the at least one trained model.

According to some embodiments, generating the radiology report by including in the radiology report at least some content from the prior report includes copying text from the prior report into the radiology report, wherein copying text from the prior report into the radiology report includes editing the text to reflect at least one value from the data regarding the patient.

According to some embodiments, the one or more systems and methods further include generating the radiology report includes populating a report template; generating the radiology report based on the data regarding the patient includes populating the report template based on at least the medical image data, the sensor data, and the content related to the notes data relating to the dictation; and generating the radiology report based on the prior patient data for the matched prior patient includes, for one or more parts of the report template not populated based on the data regarding the patient, populating the one or more parts based on content from one or more corresponding parts of the prior report.

According to some embodiments, determining whether there is a match between the data regarding the patient and the prior patient data for a prior patient includes identifying, from among one or more potential matches in the one or more prior patients, a prior patient associated with a highest measure of a match.

### DESCRIPTIONS OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates a report generation system, according to some embodiments.

FIG. 2 illustrates operations that may be performed by a base module, according to some embodiments.

FIG. 3 illustrates operations that may be performed by an imaging module, according to some embodiments.

FIG. 4 illustrates operations that may be performed by an analysis module, according to some embodiments.

FIG. 5 illustrates operations that may be performed by a report module, according to some embodiments.

FIG. 6 illustrates operations that may be performed by a review module, according to some embodiments.

FIG. 7 illustrates information that may be contained in a report database, according to some embodiments.

FIG. 8 illustrates information that may be contained in a pathology database, according to some embodiments.

FIG. 9 illustrates information that may be contained in a recommendation database, according to some embodiments.

FIG. 10 is a block diagram of a computing device with which some embodiments may operate.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Described herein are techniques for generating a medical report, such as a radiological report. In some cases, some or all of the radiological report may be generated during a medical imaging to which the report relates, such that the report is generated contemporaneously with the imaging or in real time during the imaging. Such contemporaneous generation may mean the report is generated while the patient is in an examination room in which the medical imaging is being conducted, before the medical imaging has been completed, or before the patient has been instructed to depart the examination room or the hospital. In some embodiments described herein, some or all of the radiological report may be generated automatically, without intervention from an imaging technician or from a radiologist. Such an automatic generation of radiological report content may be performed in some embodiments through comparison of information regarding a medical imaging of a patient to information regarding one or more prior medical imagings of the patient or other patients. In response to detecting a match between information regarding a medical imaging of a patient and information regarding one or more prior medical imagings, information relating to one or more such prior medical imagings may be used to generate the radiological report. In some such embodiments, the information that is used from the prior medical imaging(s) to generate the radiological report may include content of prior radiological reports relating to the prior medical imaging(s).

In some embodiments, a radiological report that is generated using techniques herein may enable replay of procedures. According to some embodiments, a replay of a procedure can involve combining preoperative, intraoperative, and/or postoperative radiology to create media and/or digital representations corresponding to the change created in radiology data based on a surgical procedure. For example, media/representations can include, but are not limited to, images, video motion graphics, slide shows, augmented reality (AR), virtual reality (VR), extended reality (XR), and/or any other type of multi-media files, objects or items, and the like, or some combination thereof. Thus, in some embodiments, the media/representation can be any type of known or to be known two-dimensional (2D) or three-dimensional (3D) rendering. For example, the replay can be a video that provides a “story board” of static images (or frames) that depict the impact of a treatment on the radiological report, which the patient can render in order to “replay” their procedure, understand what occurred, where any complications may have arisen, why/when certain annotations we added to the report, and have a more integrated understanding of how their radiological data has progressed/changed.

In some embodiments, a replay can be viewed on a traditional screen or using AR, VR and/or XR technologies. In some embodiments, data and/or metadata related to a replay can be used to train machine learning (ML) and/or artificial intelligence (AI) models for execution by a surgical robot, or for training and quality assurance evaluations after procedures. In some embodiments, a replay may alternatively comprise of documentation, such as a transcription of the procedure and/or documentation of relevant images to create an automated patient care report.

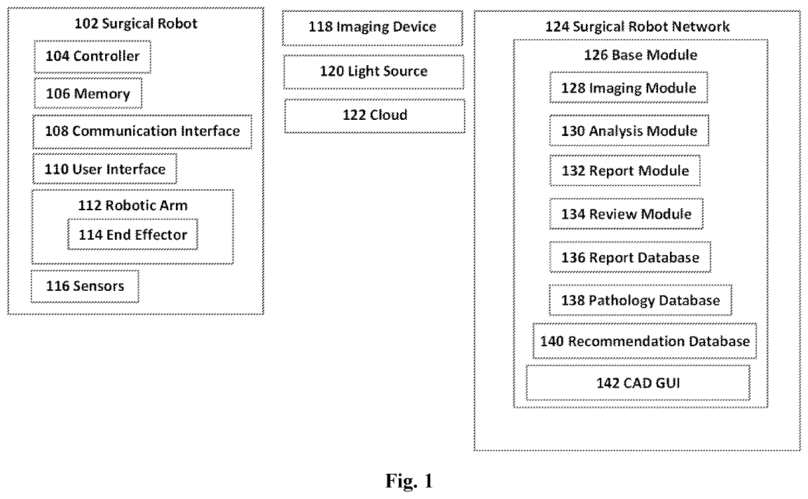

Turning to FIG. 1, in some embodiments, a system for report generation or replay may comprise a surgical robot 102. Surgical robot 102 may be a robotic system designed to assist a surgeon in performing a surgical operation on a patient. A surgical robot 102 may include a controller 104, memory 106, and at least one robotic arm 112 with an end effector 114. The surgical robot 102 may further include a user interface 110 for accepting control inputs from a user, such as a surgeon or other medical professional. The surgical robot 102 may also include a communications interface 108 for transmitting and receiving data to and from a cloud 122. In some embodiments, the communication of data to/from the cloud 122 may include communications for the purpose of training an artificial intelligence operating within the surgical robot 102. In other embodiments, the communication of data to/from the cloud 122 may additionally or alternatively include receiving remote commands from a remote user or an artificial intelligence existing external to the surgical robot 102. The surgical robot 102 may additionally comprise a plurality of sensors 116 for providing feedback to the user or an artificial intelligence operating locally and/or remotely.

Controller 104 may be a computing device comprised of a processor for performing computations and communicates with a memory 106 for storing data. The controller 104 is in communication with communications interface 108 and may further be allowed to control the at least one robotic arm 112 and end effector 114 of a surgical robot 102. The controller 104 may be a commercially available central processing unit (CPU) or graphical processing unit (GPU) or may be a proprietary, purpose-built design. More than one controller 104 may operate in tandem and may be of different types, such as a CPU and a GPU. A GPU is not restricted to only processing graphics or image data and may be used for other computations.

Memory 106 may be electronic circuitry within a computing device that stores data for usage by the controller 104. The memory 106 may comprise temporary data storage and/or persistent data storage for storing data used by the controller 104. The memory 106 may be integrated into a controller 104 or may be a discrete component. The memory 106 may be integrated into a circuit, such as soldered on component of a single board computer (SBC) or may be a removable component such as a discrete dynamic random-access memory (DRAM) stick, secure digital (SD) card, flash drive, solid state drive (SSD), magnetic hard disk drive (HDD), etc. In some embodiments, memory 106 may be part of a controller 104. Multiple types of memory 106 may be used by the surgical robot 102.

A communications interface 108 allows the surgical robot 102 to communicate with external devices and may comprise a wireless antenna and transceiver or a port for receiving a cable to facilitate a wired connection. Examples of a wired connection include ethernet, universal serial bus (USB) or a proprietary connection. A wireless communications interface 112 may include any of Wi-Fi, Bluetooth, near field communications (NFC) or a cellular communications interface such as 3G, 4G, LTE, or 5G.

The communications interface 108 may connect a user interface 110 to the surgical robot 102 or may facilitate access to a local network or a cloud network 122 to access a remote server and/or database.

A user interface 110 may enable a user to interact with a surgical robot 102 and may include a keyboard, computer mouse, trackball, joystick, wireless or wired gamepad, sliders, scroll wheels, touch screen, microphone for receiving voice commands, and/or other interaction device or any other method of interaction of a user with a surgical robot 102 not listed. The user interface 110 may accept direct inputs, such as from a joystick controlling the movement of a robotic arm or indirect inputs such as commands entered on a keyboard or touch screen such as adjusting the sensitivity of a joystick control or the speed of a robotic arm's 112 movement in response to a joystick. The user interface 110 may also comprise a screen for presenting information to the user such as patient status, imaging data, and navigation data and speakers for providing auditory feedback. The user interface 110 may also utilize haptics to provide feedback to the user. In some embodiments, the user interface 110 may additionally or alternatively comprise an augmented reality (AR) or virtual reality (VR) headset to enable a surgeon to view imagery from at least one imaging device 118 in real-time and may additionally comprise an overlay, such as highlighting the location of blood vessels. The user interface 110 may additionally comprise voice or eye tracking controls.

A robotic arm 112 is a mechanically actuated arm or lever with at least two degrees of freedom. A robotic arm 112 will typically include at least one end effector 114 or an imaging device 118 and may include both an end effector 114 and an imaging device 118. The robotic arm 112 may additionally be capable of changing the end effector 114 to facilitate multiple functions and operation of a variety of tools. The robotic arm 112 may be manually controlled or operated in an autonomous or semi-autonomous mode. A surgical robot 102 may have one robotic arm 112 or multiple robotic arms 112, each of which may be operated independently by one or more users or autonomous systems or a combination of users and autonomous systems.

An end effector 114 may be or include a tool or device for interacting with a physical object. End effector 114 may include a surgical tool intended for acting upon or within a patient. End effector 114 may additionally or alternatively be or include a gripping device for holding an object, such as a surgical tool or other object, or otherwise securing a separate surgical tool to robotic arm 112. The end effector 114 may comprise a catheter or other tool for accessing a treatment site within a patient. Similarly, the end effector 114 may relate to a deployable device, such as a stent, prior to deployment in a patient. The end effector 114 may be permanently affixed to the end of a robotic arm 112 or may be detachable, allowing for a system of interchangeable end effectors 114 which may alternatively be selected and swapped by a single robotic arm 112 or multiple robotic arms 112.

In some embodiments, the end effector 114 may be constructed of materials which intentionally absorb, reflect, or are transparent to X-Rays to facilitate the end effector's 114 visibility when viewed using angiography, fluoroscopy, or other imaging modalities, or alternatively allow X-Rays to pass through to prevent their interference in images. In some embodiments, the end effector 114 may be made to be selectively transparent to X-Rays such as by changing the profile of the end effector 114 or X-Ray absorbing or reflective components to increase or reduce their visibility to an imaging device 118.

Sensor 116 may be a measurement tool for monitoring a characteristic or metric associated with a surgical robot 102, end effector 114 or patient. A sensor 114 may be discrete or part of an array or assembly, such as integrated into a catheter. One or more of the sensors 114 may include an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, any combination thereof, etc. The sensors 116 may be integrated into the operation of the surgical robot 102 or may monitor the status of a patient. The data acquired by the sensors 116 may be used to train a machine learning algorithm used by the surgical robot 102 or artificial intelligence to control the surgical robot 102. The sensors 116 may additionally comprise an X-Ray dosimeter to monitor the intensity of X-Rays being emitted toward the patient to prevent excessive doses of radiation. The sensors 116 may be utilized to reduce the intensity of the X-Rays or reduce the duration or increase the interval in which the X-Rays are emitted toward the patient to control the dose throughout a procedure.

Imaging device 118 may be any device capable of collecting data which can be used to create an image, or a representation of a physical structure or phenomena. In some embodiments, an imaging device 118 may include any device capable of detecting sound or electromagnetic waves and assembling a visual representation of the detected waves. In other embodiments, imaging devices 118 may additionally or alternatively collect waves from any part of the electromagnetic spectrum or sounds at any range of frequencies, often as a matrix of independently acquired measurements which each representing a pixel of a two or three-dimensional image. These measurements may be taken simultaneously or in series via a scanning process or a combination of methods. Some pixels of an image produced by an imaging device 118 may be interpolated from direct measurements representing adjacent pixels in order to increase the resolution of a generated image, imaging devices 118 may receive or generate imaging data from a plurality of imaging devices 130. The plurality of imaging devices 118 may include, for example, cameras attached to the robotic arm 112, cameras mounted to the ceiling or other structure above a patient undergoing surgery in a surgical room or theater, cameras that may be mounted on a tripod or other independent mounting device, cameras that may be body worn by the surgeon or other surgical staff, cameras that may be incorporated into a wearable device, such as an augmented reality device like Google Glass, Microsoft HoloLens, etc., cameras that may be integrated into an endoscope, microscope, laparoscope, or any camera or other imaging device 118 (e.g. ultrasound) that may be present in the surgical room.

The imaging device 118 may include any algorithm or software module capable of determining qualitative or quantitative data from medical images, which may be, for example, a deep learning algorithm that has been trained on a data set of medical images.

In some embodiments, an imaging device 118 may be or include one or more devices or systems used to acquire medical imagery through magnetic resonance imaging (MRI), computed tomography (CT), X-Ray, positron emission tomography (PET), ultrasound, arthrography, angiography, fluoroscopy, myelography, and/or other imaging modalities. An imaging device 118 may acquire images in real-time or be used to create composite images or models in real-time.

A light source 120 such as surgical lights also referred to as operating light, may be an instrument that performs emission of light or other electromagnetic radiation into an area, such as into a local area or cavity of a patient undergoing surgery or other medical procedure. The light source 120 may play a role in illumination before, during, and after a medical procedure. The light source 120 may be categorized by lamp type as conventional (incandescent) and LED (light-emitting diode). The light source 120 may be categorized by mounting configuration as ceiling-mounted, wall-mounted, or floor stand. The light source 120 may be categorized by type as tungsten, quartz, and/or xenon halogens and light-emitting diodes (LEDs). The light source 120 may include sterilizable handles which allow the surgeon to adjust light positions. Some important factors affecting the light source 120 may be illumination, shadow management (cast shadows and contour shadows), the volume of light, heat management, fail-safe surgical lighting. The light source 120 may additionally refer to an emitter of particles or waves such as X-Rays, ultrasonic waves, etc. in medical imagery which may occur opposite an imaging device 118 or from the same side of the patient as the imaging device 118. In some embodiments, the light source 120 may be a part of an imaging device 118. The light source 120 can be integrated into the embodiments in a variety of manners.

A cloud 122 may be a distributed network of computers comprising servers and databases. A cloud 122 may be a private cloud 122, where access is restricted by isolating the network such as preventing external access, or by using encryption to limit access to only authorized users. Alternatively, a cloud 122 may be a public cloud 122 where access is widely available via the internet. A public cloud 122 may not be secured or may be include limited security features.

A surgical robot network 124 which may be a network connected to the surgical robot 102 in which the surgical robot may receive and send data, provide controls to a user for the surgical robot 102 through a user interface 110, and allow a user to utilize a Computer Aided Design (CAD) Graphical User Interface (GUI) 142 to design, test, and create a surgical process for a patient.

The network 124 may include a base module 126, which may include and may initiate an imaging module 128, analysis module 130, report module 132, and review module 134. Each “module” may be implemented as executable instructions stored in a memory (e.g., temporary or persistent storage) that, when executed on one or more processors or processing cores (of the same or different computing devices), cause the one or more processors to carry out the functionality that the module is designed to implement and that corresponds to the executable instructions. The executable instructions may be implemented as software code, in some embodiments, or in firmware, scripting language or other interpreted instructions, or other manner in which executable instructions may be implemented. In embodiments in which the memory in which the module is implemented with a file system, the instructions of the module may be implemented in one or more files. When executed on a computing device that executes an operating system, a module or code of a module may be executed within one or more processes and/or one or more threads of the operating system. While modules are described distinctly herein for ease of description, in some embodiments functionality that is described herein as executed by or performed by different modules may be performed by executable code that is within the same file or executed within the same process or thread.

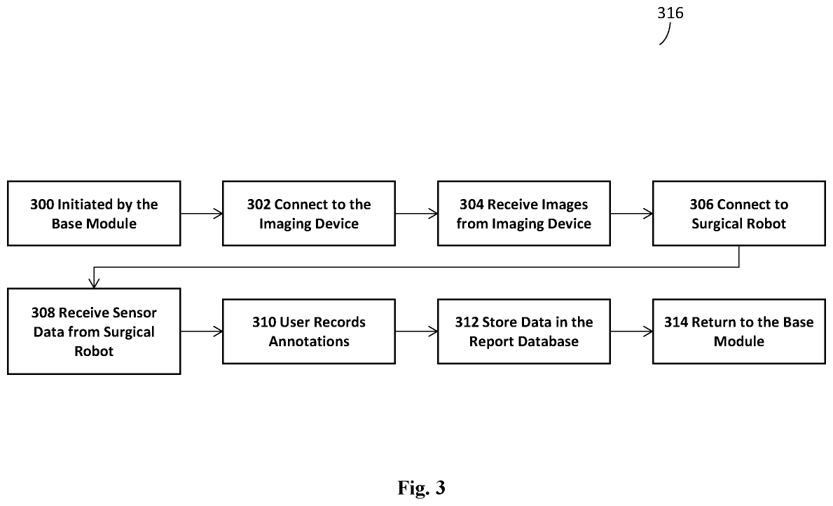

An imaging module 128 may be initiated by a base module 126 and communicate with (e.g., connect to and the exchange data with, or transmit data to and/or receive data from) an imaging device 118. In communicating with the imaging device 118, the imaging module 128 may configure and/or initiate an imaging. The imaging module 128 may receive one or more images from the imaging device 118. The imaging module 128 may also communicate with (e.g., connect to and exchange data with, or transmit data to and/or receive data from) the surgical robot 102. The imaging module 128 may receive the sensor data from the surgical robot 102. Then the user records annotations. The imaging module 128 stores the data in the report database 136. The imaging module 128 returns to the base module 126. Further detail regarding some implementations is provided below in connection with FIG. 3.

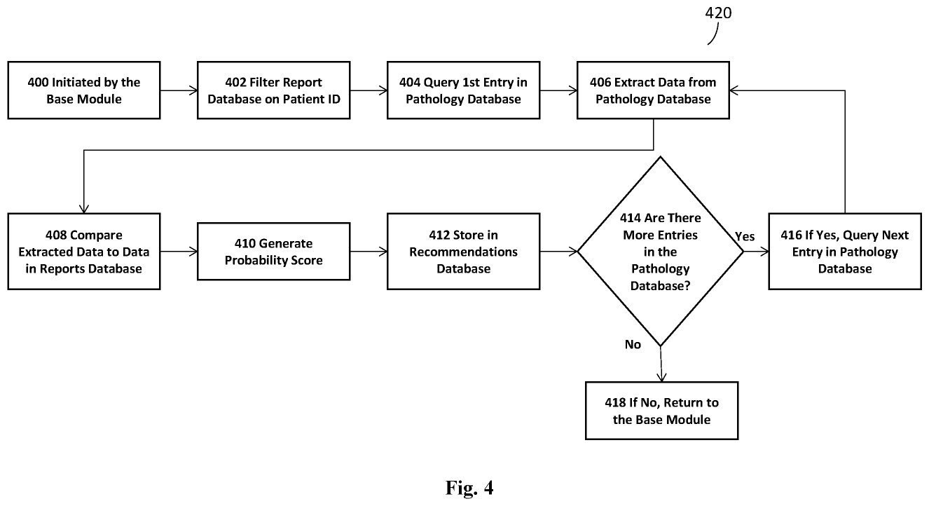

An analysis module 130 may be initiated by the base module 126, and may receive a patient ID and filter report database 136 based on that patient ID. The patient ID may be a name, number, or other identifier for a person that is used by the database 136 and/or database 138 to store information regarding the person. The analysis module 130 may also query an entry in the pathology database 138, such as the first entry in the database 138. The analysis module 130 extracts data from the pathology database 138 and compares the extracted data from the pathology database 138 to the retrieved data stored in the reports database 136 in connection with the patient ID. The analysis module 130 generates a probability score based on a result of the comparing. The analysis module 130 stores the probability score in the recommendations database 140 and determines if there are more entries in the pathology database 138. If it is determined that there are more entries in the pathology database 138 the analysis module 130 queries the next entry in the pathology database 138. If it is determined that there are no more entries in the pathology database 138 the analysis module 130 returns to the base module 126. Further detail regarding some implementations is provided below in connection with FIG. 4.

A report module 132 may be initiated by the base module 126 and may filter the report database 136 on the patient ID. The report module 132 may extract imaging data, sensor data, and annotation data from the report database 136. The report module 132 filters the recommendations database 140 on a highest probability score, such as in some embodiments based on a highest probability score determined by an analysis module 130. In some such embodiments, the report module 132 extracts from the recommendation database 140 the pathology data in a data entry associated with the highest probability score. Then the report module 132 generates the radiology report, stores the report in the reports database 136, and/or sends the report to the review module 134. The report module 132 returns to the base module 126. Further detail regarding some implementations is provided below in connection with FIG. 5.

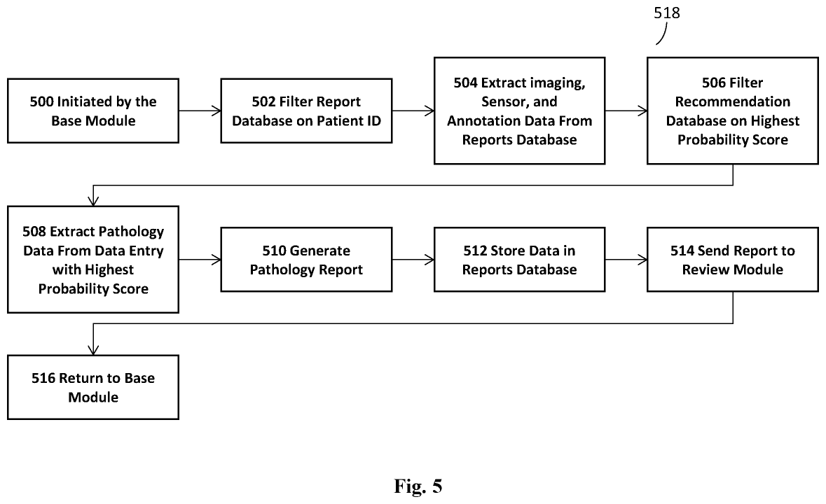

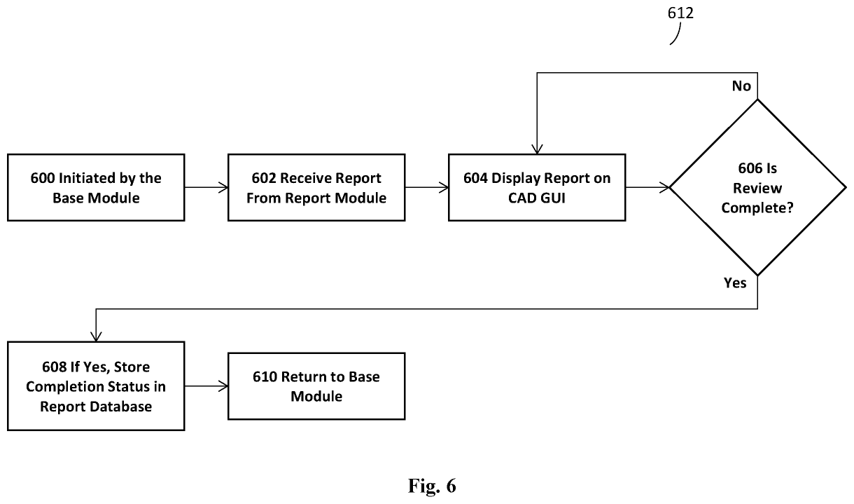

A review module 134 may be initiated by the base module 126 and may receive the report from the report module 132. The review module 134 may display the report on the CAD GUI 142. Then the review module 134 may determine if the review is complete and, if it is determined that the review is not complete, then the review module 134 may continue displaying the report on the CAD GUI 142 to allow the user to continue reviewing the report. If, however, the module 134 determines that the review is complete, the review module 134 stores a completion status in the report database 136. Then the review module 134 returns to the base module 126. Further detail regarding some implementations is provided below in connection with FIG. 6.

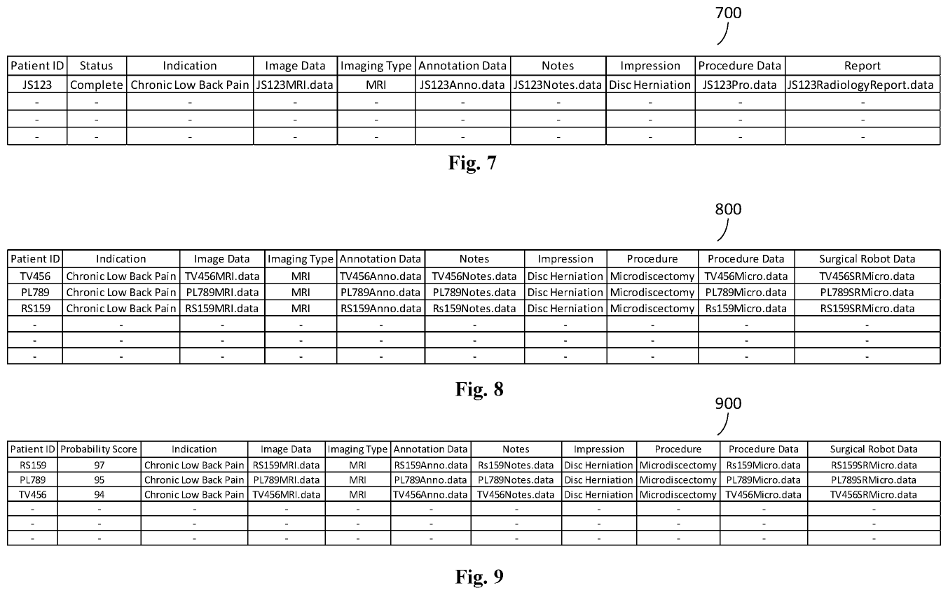

A reports database 136 may store reports created through processes described herein, including as described above and below in connection with examples of the imaging module 128, report module 132, and review module 134. The reports database 136 may be used during the analysis module 130 to compare a patient's data to historical patient data stored in the pathology database 138. The database 136 may store a variety of information for a report, such as the patient ID (e.g., “JS123”) for the patient to which the report relates, the report completion status (e.g., complete or not), a medical indication corresponding to the radiology report (e.g., chronic low back pain), image data for the report (e.g., a data file containing MRI images of the patient), imaging type used to obtain the image(s) (e.g., MRI), annotation data for the report (e.g., a data file containing a medical professional's audio recording, transcription, or written annotations), notes (e.g., a data file or notes written by a medical professional during the session in which the image(s) were taken), an impression of the image(s) (e.g., a medical professional's impression of the image(s), for example a disc herniation), procedure data (e.g., surgical procedure workflow data for the procedure performed on the patient), and the report (e.g., a radiology report stored as a data file). Further detail regarding some implementations is provided below in connection with FIG. 7.

A pathology database 138 may contain historical patient data of previous patients such as a patient's ID, an indication from a radiology report, image data, type of image data, annotation data, notes data, impression from the radiology report, procedure performed on the patient, procedure data, and surgical robot data. Further detail regarding some implementations is provided below in connection with FIG. 8.

A recommendation database 140 may contain probability scores generated by the analysis module 130, such as during an analysis in which historical patient data stored in the pathology database 138 is compared to a current patient's data to determine if a past radiology report matches a current patient scenario. In such a case, the current patient may have the same or similar procedure as the previous patient performed once reviewed and approved by a user (e.g., surgeon, physician, medical professional, etc.). The database 140 may store for a probability score entry a patient's ID, the probability score, an indication from a past radiology report, image data for the past radiology report, type of image data, annotation data, notes data, impression from the past radiology report, procedure performed on the past patient, procedure data for the past procedure, and surgical robot data for the past procedure. Further detail regarding some implementations is provided below in connection with FIG. 9.

While the term “database” is used, it should be appreciated that embodiments are not limited to implementing the databases as a relational database. Any suitable data store may be used in some embodiments, including another form of data structure or a flat file. In addition, while three databases are illustrated in the example of FIG. 9, it should be appreciated that this is for ease of description of the types of information that may be stored in some embodiments, and embodiments are not limited to separating or segmenting the storage of data described herein in any particular manner. The examples of data described herein as stored in the databases 136, 138, 140 may be stored in one or more data stores, including one or more databases.

A CAD GUI 142 may be a user interface for a computer software system to review radiology reports for patients. A GUI or guided user interface may be an interface(s) may either accept inputs from users or provide outputs to the users or may perform both the actions. In one case, a user can interact with the interface(s) using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the interface(s) may either be implemented as a Command Line Interface (CLI), a Graphical User Interface (GUI), a voice interface, or a web-based user-interface. The CAD GUI 142 may allow users, such as a surgeon, doctor, medical professional, etc. to view, review or adjust a radiology report for a patient. In some embodiments, the CAD GUI 142 may allow a user to review a radiology report and view procedures completed on past patients with radiology reports having matching information as for a current patient, which may enable the user to make decisions on the type of procedure that is appropriate for the current patient.

While not illustrated in FIG. 1, the elements of FIG. 1 may communicate with one another using suitable computer communication techniques, including by communicating via a network. The network may be any suitable combination of one or more wired and/or wireless, local- and/or wide-area communication networks, including one or more enterprise networks and/or including the Internet.

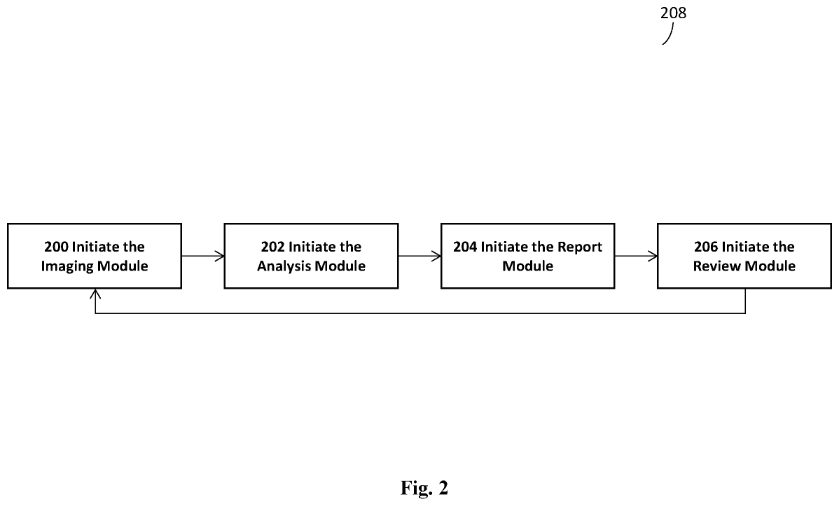

An example of how a base module 126 may operate in some embodiments will now be explained with reference to FIG. 2. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process 208 begins with the base module 126 initiating, at step 200, the imaging module 128. Initiating the imaging module 128 may include triggering execution of the imaging module 128 and/or triggering the imaging module 128 to begin operations, such as to begin the operations disclosed below in the example of FIG. 3. The imaging module 128 may be executed on the same or a different computing device than the one that is executing the base module 126. Initiating the module 128 may include configuring the module 128 to perform operations, including sending to the module 128 from the module 126 information about a patient to be imaged and/or an imaging to be performed. Once initiated, the imaging module 128 communicates with (e.g., connects to) an imaging device 118. The imaging module 128 may, upon initiation, trigger capture of images by the device 118 and may receive the images from the imaging device 118. The imaging module 128 may then communicate with (e.g., connects to) the surgical robot 102. The imaging module 128 may, upon initiation, trigger capture of sensor data by the robot 102 using one or more of its sensors, and the imaging module 128 may receive the sensor data from the surgical robot 102. The user may records annotation, and the imaging module 128 may store the data (e.g., image(s), sensor data, and/or annotations) in the report database 136, and may store the data in connection with information regarding the patient being imaged and/or the imaging, such as a patient ID or other identifier and/or a manner in which imaging was performed (e.g., type of imaging device 118, parameters with which the image was captured, etc.). In some embodiments, the imaging module 128 returns to the base module 126, which may include signaling that the imaging module 128 has been initiated, completed an operation, and/or completed all operations. The signaling from the imaging module 128 to the base module 126 may include triggering the base module 126 to continue operations. In some embodiments, the imaging module 128 may send data to the base module 126, such as the image(s) captured by the imaging device 118, the sensor data from the robot 102, and/or any other information or data, including the examples of data previously described in this paragraph or elsewhere herein in connection with the imaging module 128.

The base module 126, at step 202, initiates the analysis module 130. Initiating the analysis module 130 may include triggering execution of the analysis module 130 and/or triggering the analysis module 130 to begin operations, such as to begin the operations disclosed below in the example of FIG. 4. The analysis module 130 may be executed on the same or a different computing device than the one that is executing the base module 126 and/or the imaging module 128. In some embodiments, initiating the analysis module 130 may include configuring the analysis module 130, such as by sending to the analysis module 130 from the module 126 information regarding the patient for which an analysis is to be performed and/or regarding information that is to be analyzed by the analysis module 130. Once initiated, the analysis module 130 may filter a report database 136 based on a patient ID received by the analysis module 130 during the initiating. Filtering may be done in some embodiments by querying the database using information (e.g., the patient ID) as a search term. As another example, the module 130 may configure the database 136 to apply the filter to results of other searches received by the database 136 while so configured. Once the module 130 filters the database 136 and obtains a set of responsive information, the analysis module 130 may obtain an entry from the pathology database 138, which the analysis module 130 may perform by querying the pathology database 138. The analysis module 130 extracts data from an entry of the pathology database 138 based on the filtering and querying. The entry that is extracted relates to a prior report for a prior patient, who may be the same patient as the current patient for which the filtering of the database 136 was done or may be a different patient. The analysis module 130 may then compare the extracted data from the pathology database 138 to data stored in the reports database 136 and generates a probability score based on the comparing, where the probability score indicates a probability of match. The data stored in the reports database 136 relates to prior reports processed by the system, as discussed above, and so the comparison may be between information on a current patient and information on past patients, to find past reports that are a match to a current patient situation due to at least some of the data in the current situation and the past report being identified or meeting a criterion or criteria for being identified as a match. The probability score may indicate a degree of the match. The analysis module 130 may store the probability score, such as in the recommendations database 140. Then the analysis module 130 may determine if there are more entries in the responsive entries from the filtering of the pathology database 138. If it is determined that there are more entries in the pathology database 138 the analysis module 130 continues obtaining data for the next entry in the pathology database 138, comparing, generating a probability score, scoring, and determining whether there are more entries. The module 128 may continue this operation loop until a criterion or criteria are met, such as a threshold number of entries are processed, all entries are processed, or other criteria. Once it is determined that there are no more entries in the pathology database 138, or the criteria are otherwise satisfied, the analysis module 130 returns to the base module 126, which may include signaling that the analysis module 130 has been initiated, completed an operation, and/or completed all operations. The signaling from the module 130 to the base module 126 may include triggering the base module 126 to continue operations. In some embodiments, the analysis module 130 may send data to the base module 126, such as any of the data entries processed by the analysis module 130, any probability scores calculated by the analysis module 130, and/or any other information or data, including the examples of data previously described in this paragraph or elsewhere herein in connection with the analysis module 130.

The base module 126, at step 204, initiates the report module 132. Initiating the report module 132 may include triggering execution of the report module 132 and/or triggering the report module 132 to begin operations, such as to begin the operations disclosed below in the example of FIG. 5. The report module 132 may be executed on the same or a different computing device than the one that is executing the base module 126, imaging module 128, and/or the analysis module 130. In some embodiments, initiating the report module 132 may include configuring the report module 132, such as by sending to the report module 132 from the module 126 information regarding the patient for which a report is to be created and/or regarding information that is to be processed by the report module 132. Once initiated, the report module 132 filters the report database 136 on the patient ID for the patient being imaged or treated or otherwise for which a report is being created, which may include querying the database 136 based on the patient ID. In some embodiments, the filtering/querying may include querying for data that is not yet associated with a completed report. The report module 132 extracts from the report database 136 imaging data, sensor data, and annotation data for a report to be created. The report module 132 also filters the recommendations database 140 on probability score, such as by querying the recommendations database 140 for a highest probability score or based on another criterion or criteria. In response to the filtering/querying, the report module 132 may obtain from the database 140 an identification of an entry in the report database 136 for a prior report that is associated with the probability score, such as the prior report having the highest degree of match to a current situation for a current patient. The report module 132 extracts from the database 136 and/or database 140 data, such as pathology data, from the data entry with the probability score. Then the report module 132 generates the radiology report and stores the report in the reports database 136. The report module 132 may additionally or alternatively send the report to the review module 134. The report module 132 returns to the base module 126, which may include signaling that the report module 132 has been initiated, completed an operation, and/or completed all operations. The signaling from the module 132 to the base module 126 may include triggering the base module 126 to continue operations. In some embodiments, the report module 132 may send data to the base module 126, such as any of the data entries processed by the report module 132, the report generated by the report module 132, and/or any other information or data, including the examples of data previously described in this paragraph or elsewhere herein in connection with the report module 132.

According to some embodiments, generation of the report, as discussed herein, can be based on execution of a machine learning and/or artificial intelligence algorithm, such as, an image recognition algorithm, for example. In some embodiments, such implementations can involve, but are not limited to, analysis of pixels characteristics, features and/or attributes, where such characteristics, features and/or attributes can correspond to, but are not limited to, brightness, hue, saturation, or some other pixel measurement, as one example of image recognition.

The base module 126, at step 206, initiates the review module 134. Initiating the review module 134 may include triggering execution of the review module 134 and/or triggering the review module 134 to begin operations, such as to begin the operations disclosed below in the example of FIG. 6. The review module 134 may be executed on the same or a different computing device than the one that is executing the base module 126, imaging module 128, the analysis module 130, and/or the report module 132. In some embodiments, initiating the review module 134 may include configuring the review module 134, such as by sending to the review module 134 from the module 126 information regarding the patient for which a report is to be reviewed, the report to be reviewed, and/or regarding information that is to be processed by the review module 134. Once initiated, the review module 134 may receive the report from the report module 132 or otherwise obtain the report, such as from the base module 126 or the report database 136. The review module 134 may output the report for display to a user, such as via the CAD GUI 142. The review module 134 may determine if review of the report is complete, such as by monitoring user interaction to determine whether the user has provided an express input indicating completion of review, if all portions of the report have been displayed to the user, or if the user's interactions indicate completion such as by pausing interaction. If it is determined that the review is not complete, then the review module 134 continues outputting the report for display on the CAD GUI 142, or otherwise does not cease outputting or presentation of the report, to allow the user to continue reviewing the report. If it determines that the review is complete, the review module 134 stores a completion status in the report database 136. Then the review module 134 returns to the base module 126, which may include signaling that the review module 134 has been initiated, completed an operation, and/or completed all operations. The signaling from the module 134 to the base module 126 may include triggering the base module 126 to continue operations. In some embodiments, the review module 134 may send data to the base module 126, such as an identification of or other information regarding the report output by the review module 134, information regarding the patient to which the report relates, and/or any other information or data, including the examples of data previously described in this paragraph or elsewhere herein in connection with the review module 134.

Examples of functioning of the imaging module 128 will now be explained with reference to FIG. 3. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process 316 of FIG. 3 begins with the imaging module 128 being initiated at step 300, such as by a user, by the base module 126 as discussed above, or initiated in another manner. Initiating the imaging module 128 may include triggering execution of the imaging module 128 and/or triggering the imaging module 128 to begin operations, such as to begin the operations disclosed in this example of FIG. 3. Initiating the module 128 may include configuring the module 128 to perform operations, including receiving at the module 128 from a user or from the module 126 information about a patient to be imaged and/or an imaging to be performed. Information regarding the patient may include an identifier for the patient (name, alphanumeric identifier, or other identifier), demographic information for the patient (age, ethnicity, or other information), medical status or medical history (e.g., prior conditions or surgeries), or other information regarding the patient. The information regarding the imaging to be performed may include information regarding a type of imaging to be performed, equipment with which the imaging is to be performed, anatomy to be imaged and/or an indication of what (if any) suspected medical condition the anatomy is being imaged for to determine whether the condition is present, parameters with which to configure the equipment to perform the imaging or otherwise instructions to issue to the equipment, or other suitable information.

The imaging module 128 connects, at step 302, to the imaging device 118. As part of connecting to the imaging device 118, the module 128 may communicate information to the imaging device 118, such as to configure the imaging device 118 and/or trigger image capture by the imaging device 118. The information that is communicated may include information regarding the patient to be imaged and/or an imaging to be performed, examples of which were discussed immediately above. As mentioned above in connection with FIG. 1, imaging device 118 may be or include any device capable of collecting data which can be used to create an image, or a representation of a physical structure or phenomena. Imaging techniques that the imaging device 118 may use include magnetic resonance imaging (MRI), computed tomography (CT), X-Ray, positron emission tomography (PET), ultrasound, arthrography, angiography, fluoroscopy, myelography, and/or other medical imaging techniques.

The imaging module 128, at step 304, receives one or more images from the imaging device 118. For example, the imaging module 128 receives the image(s) from the imaging device 118. The images may be of a patient, such as of a patient identified in information transmitted to the imaging device 118. In a case that multiple images are captured and received, the images may depict the same or different aspects of anatomy of the patient, and in a case that the same anatomy is shown, may depict the anatomy from different perspectives, under different imaging conditions or configurations, or otherwise vary in how the anatomy was captured or is depicted. Embodiments are not limited to operating with any particular form of imaging.

In addition to, or as an alternative to, receiving the medical image(s) in block 304, the imaging module 128 may receive notes from one or more radiologists regarding the medical image(s). The notes from the radiologists may include notes entered via a user interface, such as user interface 110 of FIG. 1. The notes may include any suitable remarks of a radiologist regarding what is depicted in the image(s), including impressions, diagnoses, identifications of what is depicted, dimensions of anatomical features depicted, or other content.

In the example of FIG. 3, the imaging module 128 next, at step 306, connects to a surgical robot 102. As part of connecting to the surgical robot 102, the module 128 may communicate information to the surgical robot 102, such as to configure the surgical robot 102 and/or trigger operation of the surgical robot 102. The information that is communicated may include information regarding the patient on which surgery is to be performed and/or a procedure to be performed on the patient, examples of which information are discussed herein.

For example, the imaging module 128 may connect to a surgical robot 102 that is a robotic system designed to assist a surgeon in performing a surgical operation on the patient. A surgical robot 102 may include a controller 104, memory 106, and at least one robotic arm 112 with an end effector 114. The surgical robot 102 may further include a user interface 110 for accepting control inputs from a user, such as a surgeon or other medical professional and a communications interface 108 for transmitting and receiving data to and from a cloud 122 for the purpose of training an artificial intelligence operating within the surgical robot or receiving remote commands from a remote user or an artificial intelligence existing external to the surgical robot 102. The surgical robot 102 may additionally comprise a plurality of sensors 116 for providing feedback to the user or an artificial intelligence. Examples of components and configurations of a robot 102 are described above in connection with FIG. 1.

As part of connecting to the surgical robot 102 in step 306, the imaging module 128 may trigger the surgical robot 102 to operate one or more sensors of the robot 102 to collect sensor data. Examples of sensor and sensor data are described above. Such sensor data may include information regarding the patient, such as physiological or biological information regarding the patient collected by measuring the patient or anatomy of the patient. In some embodiments, in step 306, the imaging module 128 may specify the sensor(s) of the robot 102 to be operated and/or indicate what sensor data or characteristics/information of the patient is to be collected, such as by requesting information from the surgical robot 102. In some embodiments, such sensors and sensor data may include an electrophysiologic sensor, a temperature sensor, a thermal gradient sensor, a barometer, an altimeter, an accelerometer, a gyroscope, a humidity sensor, a magnetometer, an inclinometer, an oximeter, a colorimetric monitor, a sweat analyte sensor, a galvanic skin response sensor, an interfacial pressure sensor, a flow sensor, a stretch sensor, a microphone, any combination thereof, etc., that are attached, embedded, or otherwise included in the surgical robot 102.

The imaging module 128, at step 308, receives the sensor data from the surgical robot 102. For example, the imaging module 128 receives sensor data from the surgical robot 102, such as data from the aforementioned sensors. In some embodiments, the surgical robot 102 may have an imaging device 118 attached or embedded in an end effector 114 that captures medical images, and in such cases the step 308 of receiving sensor data may include receiving medical images.

In some embodiments, in step 308, the imaging module 128 may receive information regarding a procedure performed on a patient, such as a surgical procedure performed. This may be a surgical procedure performed using surgical robot 102. The information regarding the procedure may include information identifying the procedure, describing how the procedure was conducted, an outcome of the procedure, any complications that arose during the procedure, or other content describing the procedure and how it was conducted. Such information may have been automatically collected by the surgical robot 102 through one or more sensors or recorded automatically during operation of the robot 102, and/or may have been input by a user (e.g., a surgeon, nurse, or other clinician) via a user interface 110 of the robot 102.

At step 310, the imaging module 128 receives user annotations. For example, a user may be a physician, surgeon, medical professional, radiologist, etc. in which a note of explanation or comment added to the medical images, procedure, etc. is included and recorded by a microphone or written as text. In some embodiments, the surgical robot 102 or imaging device 118 may include a microphone in order to capture the user's annotations.

The imaging module 128, at step 312, stores some or all of the data received in steps 302-310 or otherwise received in the report database 136. For example, the imaging module 128 stores the received data from the imaging device 118, surgical robot 102, and the user in the report database 136. In some embodiments, the information may be stored in report database 136 in connection with information regarding a patient, such as an identifier for a patient. The information may be stored in any suitable format, including formatted as a report, formatted as data outside of a report context, or formatted as a partial report.

Once the information is stored in the report database 136, the imaging module 128 at step 314 returns processing to the base module 126. Examples of manners in which the operation of step 314 may be carried out are described above in connection with FIG. 2.

Functioning of the analysis module 130 will now be explained with reference to FIG. 4. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process 420 of FIG. 4 begins with the analysis module 130 being initiated at step 400, such as by a user, by the base module 126 as discussed above, or initiated in another manner. Initiating the analysis module 130 may include triggering execution of the analysis module 130 and/or triggering the analysis module 130 to begin operations, such as to begin the operations disclosed in this example of FIG. 4. Initiating the module 130 may include configuring the module 130 to perform operations, including receiving at the module 130 from a user or from the module 126 information about a patient for which information is to be analyzed and/or information to be analyzed. Information regarding the patient may include an identifier for the patient (name, alphanumeric identifier, or other identifier), demographic information for the patient (age, ethnicity, or other information), medical status or medical history (e.g., prior conditions or surgeries), or other information regarding the patient.

The analysis module 130, at step 402, filters the report database 136 based on information regarding a patient, such as a patient ID or other identifier for a patient. For example, the analysis module 130 may filter the report database 136 on the patient ID (e.g., an alphanumeric identifier such as “JS123”) to identify from the database a patient's data that was stored in the database 136, such as by the imaging module 128 following collection by the imaging module 128. Filtering may be done in some embodiments by querying the database using information (e.g., the patient ID) as a search term. As another example, the module 130 may configure the database 136 to apply the filter to results of other searches received by the database 136 while so configured. Once the filtering is done, the analysis module 130 obtains from the database 136 information on a patient for which a report is to be generated, which may be a patient for which an imaging was conducted and/or a procedure performed and for which a report has not yet been generated. In some embodiments, in step 402, the report database 136 may indicate for an entry whether a report has been created or finalized for information in the database 136. In step 402, the analysis module 130 may retrieve information for an entry indicated to be not yet in a report or in a finalized report and associated with the patient ID for which a report is to be generated.

Next, at step 404, the analysis module 130 queries the pathology database 138 to obtain information on past reports associated with previous patients. There may be multiple entries in the pathology database 138, and in step 404 the analysis module 130 obtains a first of the entries in the pathology database 138. This first entry may be the first retrieved, not necessarily the first stored in time or first in an organization of data in the database 138. For example, the analysis module 130 may query the pathology database and obtain a data entry for a patient, which may in this example be associated with the patient ID “TV456.” Though, it should be appreciated that embodiments are not limited to obtaining an entry for a different patient. In some cases, the pathology database 138 may store information for a past report of the same patient and the past report for that patient may be obtained in the process 420 of FIG. 4.

The analysis module 130, at step 406, extracts the data from the pathology database 138 for the data entry obtained in step 404. For example, the analysis module 130 extracts the data for the queried data entry, such as the indication, image data, imaging type, annotation data, notes, impression, procedure, procedure data, and/or surgical robot data for the entry for the patient with the ID TV456. It should be appreciated that this is a set of illustrative data that may, in some embodiments, be stored in the pathology database 138 and that embodiments are not limited to storing any or all of this information. In embodiments that store at least some of this information, embodiments are not limited to storing the same types of information for every entry, and different entries may store different information.

In step 408, the analysis module 130 compares the extracted data from the pathology database 138 for a prior patient (the information obtained in steps 404, 406) to the data previously obtained from the reports database 136 (in step 402) for the patient for which a report is to be created. For example, the entry retrieved from the pathology database 138 for the prior patient and the data obtained from the reports database 136 for the patient for which a report is to be created may each include one or more of an indication, imaging data (such as MRI images), the imaging type (such as an MRI), annotation data, notes data, radiologist impression, or other data elements. In such a case, the information between the two entries may be compared to determine if there are similarities between the two patients' data.

For example, if each entry contains one or more items of information (e.g., indication, annotation, notes, etc.), the analysis module 130 may determine any same items of information included in both entries (e.g., both include an indication), then compare each pair of items of information to determine whether content or values for the items match. Items of information may match when they are identical. In some embodiments, items of information may match when they are not identical but include content that meets one or more criteria defining a match.

As another example, different items of information may be compared between the two entries, such as by determining whether key words or concepts from an entry for a current patient appear in the prior entry for a past patient, even if in different items of information. For example, the analysis module 130 may determine whether a symptom identified in an indication field of a current patient entry and/or an identification of anatomy under examination in a medical image appear in items of information in the past patient entry.

A criterion for defining a match may vary between types of values or content included in an item of information. For a numeric value, for example, a match may be identified when numeric values are within a threshold amount of one another, such as an absolute number threshold or a relative threshold like a percentage. For a textual value, as another example, a match may be identified when text includes more than a threshold number of identical words, when a semantic interpretation indicates that the text matches to more than a threshold amount (absolute number or percentage) of the same concepts in an ontology, or other manner of determining a match between text. For image values, as another example, a match may be identified when objects are identified in both images, such as that both images depict the same underlying anatomy and additional objects (e.g., aberrant tissues like tumors, deposits of biological material like blood clots, foreign bodies, etc.) are identified in or alongside the anatomy. Embodiments are not limited to a specific manner in which a match between values for items of information, or a match between entries, are determined. Matching entries may be identical or similar.

Below is provided an illustrative example of how the analysis module 130 may compare data for entries.

As one example, the analysis module 130 may compare the extracted data, such as the current patient's image data, to the notes data stored in the pathology database 138 to find recommendations for the impression, or diagnosis, and procedure for the current patient. For example, the pathology database 138 may be filtered on the current patient's indication, such as chronic low back pain, and the current patient's image data, such as an MRI, is compared to the historical notes data stored in the pathology database 138. The analysis module 130 performs correlations on the data to find the highly correlated notes data. For example, if the current patient's image data is of a potential L3-L4 disc herniation and the historical notes data mentions L3-L4 disc herniation, the data would be highly correlated. Then the analysis module 130 may compare the highly correlated notes data to the impression, or diagnosis, data and procedure data to determine the impression and procedure recommendation for the current patient. For example, the highly correlated notes data, which mentions L3-L4 disc herniation, is compared to the historical impression and procedure data to find the highly correlated data, which would be impressions and procedures for a L3-L4 disc herniation. The results may be that the highly correlated impression and procedure data may be that the impression is a disc herniation between the L3-L4 vertebrae, and the procedure would be a microdiscectomy to fix the disc herniation. The impression and procedure would be stored in the recommendations database 140 to be extracted in the process described in the reports module 132 for the impression, disc herniation between the L3-L4 vertebrae, and the procedure, a microdiscectomy, and be added in the generated radiology report to diagnosis the current patient and recommend the appropriate procedure to fix the disc herniation.

The analysis module 130, at step 410, generates a probability score based on a result of the comparing of step 408. Such a probability may indicate a probability that the two entries are a correct match. With such a probability score, if the comparing of step 408 identifies more matching content between the entries, a probability score may be higher.

For example, the analysis module 130 may generate a probability score in step 410 by determining a percentage of how much of a match the two data entries have been determined to be. For example, if the indication, such as chronic low back pain, is a match or the exact same then the indication, stored in the databases, would have a 100% probability of being a match. If the MRI images are both of the lumbar region of the spine and the five discs that are captured are a match, such as between L1-L2, L2-L3, L4-5, are all normally separated by a fully intact disc and the image of the disc between L3-L4 is protruding, then the images would have 100% probability of being a match. If the impressions stored for the two databases mention a L3-L4 disc herniation, then the impressions stored in the databases would have 100% probability of being a match. The various percentages could then be averaged to determine a total probability, such as 100%, and the score generated would be 100 and the probability score, along with the data entry from the pathology database 138, would be stored in the recommendations database 140.

In some embodiments, the comparing of block 408 and the generation of the probability score in block 410 may be performed using an artificial intelligence or machine learning system, and the probability score may be a confidence value or correlation coefficient generated by the system. In some such embodiments, the pathology database 138 is filtered on the indication stored in the report database 136 and the first parameter from the entry in the pathology database 138 is selected, such as the age of the patients, and then correlations are performed on all the other parameters stored in the pathology database 138, such as the image data of where the disc herniation is located on the spine, such as between L1-L2, L2-L3, etc., the annotation data of where the disc herniation has been identified by the radiologist, etc. An example of highly correlated data may the age of the patient and the location of the disc herniation and then the highly correlated data is compared to the current patient's data, such as the same age, and the data entries with the patient's same age are extracted and given a probability score through the correlation coefficient, such as a correlation coefficient of 0.95 would be a probability score of 95.

According to some embodiments, such comparison and/or probability determinations can be performed via any type of known or to be known machine learning and/or artificial intelligence algorithm, technique and/or model, such as, for example, computer vision, feature vector analysis, decision trees, boosting, support-vector machines, neural networks, nearest neighbor algorithms, Naive Bayes, bagging, random forests, logistic regression, and the like. For example, as discussed above, the comparison/probability can be performed via an image recognition algorithm, as discussed herein.

According to some embodiments, the determined probability can be associated with and/or based on true and false values. For example, 0.6667 can be associated with true and 0.3333 can be associated with false. In some embodiments, such true/false values can be referred to in terms of a confidence interval. In some embodiments, additional and/or alternative scoring can further be utilized, such as, but not limited to, using a fixed criteria in accordance with a rubric to determine an objective score. In some embodiments, further implementations may involve applying a weighting based upon the significance of one or more criteria. In some embodiments, a threshold probability, score, confidence interval, etc. can be used to determine significance or relevance, which can impact how the disclosed scoring is determined.

The analysis module 130, at step 412, stores the probability score for the entry in the recommendations database 140. For example, the probability score would be stored in the recommendation database 140 along with an identification of the data entry from the pathology database 138. The probability score may, in some embodiments, also be stored in association with a patient ID and/or identification for the entry from the reports database 13 for which the comparison was done and for which a report is to be generated, so that the probability scores are associated with and can be later retrieved for that patient/report. The storing enables subsequent identification and retrieval of the entry for generating a radiology report by the report module 132 for the current patient, for subsequent review the current patient's radiology report through the CAD GUI 142 in the review module 134. In some embodiments, data for the entry from the pathology database 138, such as all or some of the data from the database 138, may be stored in the entry in the recommendations database 140 in step 412. In some embodiments, the probability score and identification of the entry may be stored for all entries analyzed by the analysis module 130 in steps 406-412, which may be all entries stored in the pathology database 138. In other embodiments, the analysis module 130 may store only in step 412 the probability scores and entry identifications for those entries where the probability score satisfies one or more criteria, such as by being above a threshold value. This may reduce the amount of data stored, including for matches that may not be correct.

The analysis module 130 determines, at step 414, if there are other entries in the pathology database 138 to be reviewed through the process 420 of FIG. 4, in particular through steps 406-412. If there are more data entries stored in the pathology database 138, the analysis module 130 queries the next data entry to be analyzed in step 416, obtains the next entry in the pathology database 138, and returns to step 406 to analyze the data for that entry as discussed above. If it is determined that there are no more entries in the pathology database 138, the analysis module 130 returns, at step 418, to the base module 126. Examples of manners in which the operation of step 418 may be carried out are described above in connection with FIG. 2.

Functioning of the report module 132 will now be explained with reference to FIG. 5. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process 518 begins with the report module 132 being initiated at step 500, such as by a user, by the base module 126, or in another manner. Initiating the report module 132 may include triggering execution of the report module 132 and/or triggering the report module 132 to begin operations, such as to begin the operations disclosed in this example of FIG. 5. Initiating the module 132 may include configuring the module 132 to perform operations, including receiving at the module 132 from a user or from the module 126 information about a patient for which a report is to be generated and/or information describing the imaging, procedure, or other occurrence for which the report is to be generated. Information regarding the patient may include an identifier for the patient (name, alphanumeric identifier, or other identifier), demographic information for the patient (age, ethnicity, or other information), medical status or medical history (e.g., prior conditions or surgeries), or other information regarding the patient.

The report module 132, at step 502, filters the report database 136 on the patient ID for the patient for which the report is to be generated. To continue the example used above, the report module 132 filters the report database 136 on the patient ID “JS123.” Filtering may be done in some embodiments by querying the database 136 using information (e.g., the patient ID) as a search term. As another example, the module 130 may configure the database 136 to apply the filter to results of other searches received by the database 136 while so configured. Once the filtering is done, the analysis module 130 obtains from the database 136 information on a patient for which a report is to be generated, which may be a patient for which an imaging was conducted and/or a procedure performed and for which a report has not yet been generated. In some embodiments, in step 502, the report database 136 may indicate for an entry whether a report has been created or finalized for information in the database 136. In step 502, the analysis module 130 may retrieve information for an entry indicated to be not yet in a report or in a finalized report, and associated with the patient ID for which a report is to be generated.

The report module 132, at step 504, extracts from the report database 136 data for that patient and for which a report is to be generated. The extracting of step 504 may include obtaining data as a result of a query of step 502, or obtaining data in response to a query following the filtering of step 502, or otherwise obtaining data that satisfies the filter of step 502. Any suitable data may be obtained for use by the report module 132, as embodiments are not limited in this respect. For example, the module 132 may obtain from the database 136 imaging data, sensor data, annotation data, and/or other data regarding the patient from the report database 136. For example, the report module 132 extracts imaging data, such as the MRI images, sensor data, such as notes from the imaging procedure, and annotation data, such as a data file containing radiologist annotations. The data that is obtained in step 504 may be data that will be included in a report or is to be used to generate information to be included in the report.

The report module 132, at step 506, filters the recommendations database 140 based on the probability score. Filtering may be done in some embodiments by querying the database 140 using one or more parameters (e.g., probability score) as a search term and/or as an indication of how results should be sorted (e.g., highest to lowest probability score). The filtering may, in some embodiments, also be done based on the patient ID and/or entry from the reports database 136 for which a report is to be generated, to ensure the probability scores that are obtained are for the current patient/report being processed by the report module 132. As another example, the module 130 may configure the database 140 to apply the filter to results of other searches received by the database 140 while so configured. Once the filtering is done, the analysis module 130 obtains from the database 140 information on entries for prior reports and for prior patients that are each associated with a probability score, such as a probability score generated as discussed above. For example, the report module 132 may query the recommendation database 140 for the entry associated with the highest probability score, such as highest to lowest. The database 140 may contain the patient's ID, an identifier for the report, and the probability score. In some embodiments, the database 140 may additionally include information from the entry to which the probability score relates, such as the indication from the radiology report, the image data, the type of image data, the annotation data, the notes data, the impression from the radiology report, the procedure performed on the patient, the procedure data, and the surgical robot data.

While in some embodiments, the entry and probability score identified by the report module 132 in step 506 may be the entry associated with the highest probability score, embodiments are not so limited. In other embodiments, other criteria may be evaluated. For example, if two or more entries have the same probability score or have probability scores within a threshold amount of one another, or if two or more entries each have a probability score above a threshold, the report module 132 may consider that each of the entries is a potential candidate for a match and may evaluate criteria in addition to or as an alternative to the probability score. For example, the module 132 may evaluate time at which a report was generated and select the report soonest in time to the current time, so the entry is chosen that is most similar to the current one. As another example, if any of the potential candidates relates to the same patient as the current patient under analysis, that entry may be chosen. As a further example, if any of the potential candidates is for the same clinician (e.g., surgeon, radiologist, primary care doctor, or other clinician), then that entry may be chosen. As another example, some items of information contained in reports may be weighted higher than others, such as indications, impressions, or other notes, and the potential candidates may be reevaluated with weights assigned to those items to calculate a new probability score (using a process similar to that discussed above in connection with FIG. 4) and an entry chosen based on the new probability score. Embodiments are not limited to a manner in which an entry is chosen.

The report module 132, at step 508, extracts pathology data from the selected data entry, which as discussed above in some embodiments may be the entry associated with the highest probability score in the recommendation database 140. For example, data entry may contain the patient's ID, the probability score, the indication from the radiology report, the image data, the type of image data, the annotation data, the notes data, the impression from the radiology report, the procedure performed on the patient, the procedure data, and the surgical robot data.

Then the report module 132, at step 510, generates a radiology report using both the information obtained in step 504 for the current patient and the information obtained for the entry selected in steps 506, 508. For example, the report module 132 generates the radiology report by filling in the data for the report using the patient's extracted data and the data from the data entry with the highest probability score in the recommendation database 140. The report may be used to populate a report template, in some embodiments. For example, the module 132 may populate the template first using information from the current patient entry that was obtained in step 504, such as the current patient's indication, such as chronic low back pain, the imaging data, the type of imaging performed, such as an MRI, using the radiologist's annotations and notes for the findings section in the report, etc. from the data stored in the reports database 136. If there are any remaining sections in the template that have not yet been populated using the information from the information obtained in step 504, then the report module 132 may populate one or more of those sections using information for the matched prior entry that was obtained in steps 506, 508. For example, the remaining sections of the report may be completed by using the data entry identified using the recommendation database 140. In such a case, the information from the data entry identified using the recommendation database 140 may be copied directly into a section of the report template being populated, and/or may have text copied and then edited to reflect values for the current patient. The report module 132 may thus, in copying over content from the prior report, change identifiers, values, or other information to be consisted with the information included in the report database 136 for the current patient. As one particular example of including information in the report from the prior report, for a prior report that the analysis module 130 found to include matching imaging data for an impression such as a disc herniation, the report module 132 may copy into the report being generated the procedure that was recommended for the prior patient, such as a microdiscectomy, so as to prepare content of the radiology report being generated. In some embodiments, procedure data and surgical robot data performed for the historical patient may also be obtained and included in the report, where the current patient is to have the same procedure performed.

The report module 132, at step 512, stores the generated report in the reports database 136. For example, the report module 132 stores the report in the report database 136 as a data file. According to some embodiments, a generated report can include, but is not limited to, text, images, multi-media, video, annotations, and the like. For example, in some embodiments, a report can include embedded figures, embedded comparison figures (e.g., historical versus actual, for example), automatically generated annotations (e.g., explanation of a figures meaning, technical versus layman, risk/concern level, and the like, for example), patient/provider information, report metadata (e.g., date, time and devices in use, for example), probability results (e.g., a percentage of people had full recovery, a percentage had partial recovery, and the like, for example), and the like, or some combination thereof. In step 514, the report module 132 may send the report to the review module 134 for review by a user, such as by sending the report or an identifier for the report in the report database 136. For example, the report may include current patient's indication, such as chronic low back pain, the imaging data, the type of imaging performed, such as an MRI, the findings, such as the findings during the imaging procedure, the impression data, such as a disc herniation, the recommended procedure for the patient to have performed, such as a microdiscectomy. The report module 132, at step 518, returns to the base module 126. Examples of manners in which the operation of step 518 may be carried out are described above in connection with FIG. 2.

According to some embodiments, for example, a report (e.g., radiological report) can be created in any type of known or to be known format and can include any type of known or to be known types of content. For example, the report can be a printable digital file, can include images, be based on automatically generated text and/or dictation, can include video playback and/or computer generated graphics, and the like, or some combination thereof. Accordingly, in some embodiments, a report may be viewed as, but not limited to, a paper document, a digital document, an interactive webpage, and may additionally include 2D/3D representations viewable in a VR environment. In some embodiments, the report may include data allowing for an AR and/or XR overlay upon the physical patient and/or a digital representation of the patient. As mentioned above, the report may comprise recommended procedures, but may further include documentation and/or imagery describing or depicting the procedure. In some embodiments, while such reports are written for technical personal, the report may include multiple versions and/or translations of technical terms into common language to make the documentation easier to understand. In some embodiments, a report may include contact and/or demographic information, and/or can be automatically shared with relevant parties, such as primary care providers, specialists assigned to treatment (e.g., surgeons, physical therapists, the patient, patient caregiver, and the like), and the like.

Functioning of the review module 134 will now be explained with reference to FIG. 6. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process 612 begins with the review module 134 being initiated at step 600, such as by a user, by the base module 126, or in another manner. In some embodiments, the review module 134 may be initiated to display a medical image captured during a medical imaging of a patient to a clinician, such as a radiologist. In some such embodiments, this may be the first time that the medical image is presented to a clinician following capture of the image, and may be done contemporaneous with the imaging of the patient. For example, the review module 134 may be initiated while the patient is still being imaged, while the patient is still in the imaging room or suite, or before the patient has been instructed to or has left the hospital or other medical facility where the imaging has taken place. Accordingly, the operations of FIGS. 2-6 may each take place to generate the report contemporaneous with the imaging, such as in real-time during the imaging. In some such embodiments, rather than presenting the medical image to a clinician for the clinician to be able to generate a report for the medical imaging, through the processes of FIGS. 2-5 a proposed report may be generated and provided to the review module 134 and presented to the clinician as a proposed report for the imaging, for the clinician to review, edit as appropriate, and approve.

The review module 134, at step 602, receives the report from the report module 132. For example, the report may include the current patient's indication, such as chronic low back pain, the imaging data, the type of imaging performed, such as an MRI, the findings, such as the findings during the imaging procedure, the impression data, such as a disc herniation, the recommended procedure for the patient to have performed, such as a microdiscectomy.

The review module 134, at step 604, displays the report on the CAD GUI 142. For example, the report is displayed on the CAD GUI 142 for the user to review the radiology report generated by the report module 132. In some other embodiments, rather than the review module 134 displaying the report, the review module 134 may output the report for display by another element, such as by outputting the report over a network or outputting the report via an interprocess communication mechanism.

Then the review module 134 determines, at step 606, if the review is complete, such as by monitoring user interaction to determine whether the user has provided an express input indicating completion of review, if all portions of the report have been displayed to the user, or if the user's interactions indicate completion such as by pausing interaction. If it is determined that the review is not complete, then the review module 134 continues displaying or outputting for display, or otherwise does not cease display of, the report on the CAD GUI 142 to allow the user to continue reviewing the report. In some embodiments, the user may approve of the review in order to store a completion status in the report database 136 to present the findings and course of action to the patient. In some such embodiments, the user may indicate that review is complete by approving of the review. The user may indicate that review is complete in any suitable manner, such as by interacting with the CAD GUI 142 to provide an explicit input regarding completion (e.g., press a complete button), closing the report, or other interaction.

If it is determined that the review is complete the review module 134 stores, at step 608, a completion status in the report database 136. For example, the user may approve of the review in order to store a completion status in the report database 136 to present the findings and course of action to the patient. Then the review module 134 returns, at step 612, to the base module 126. Examples of manners in which the operation of step 612 may be carried out are described above in connection with FIG. 2.

An example of a report database 136 will now be explained with reference to FIG. 7. The report database 136 may be created through the processes described in the imaging module 128, report module 132, and review module 134 and may be used during the analysis module 130 to compare the patient's data to historical patient data stored in the pathology database 138. In the example of FIG. 7, the database contains the patient ID (e.g., an alphanumeric identifier such as “JS123”), the report completion status (such as complete or not), the indication of the radiology report (such as chronic low back pain), the image data (such as a data file containing MRI or other medical images of the patient), the imaging type (such as an MRI or other imaging modality), the annotation data (such as a data file containing a medical professional's audio recording, a transcription of audio of speech, or written annotations), notes (such as a data file such as the notes written by a medical professional during a session in which the images were obtained, ordered, and/or reviewed), the impression (such as a medical professional's impression of the images, for example a disc herniation), and procedure data (such as surgical procedure workflow data related to a procedure performed on the patient), and/or a report (such as a radiology report stored as a data file). In some embodiments, the image data may be an MRI obtained through a medical imaging technique that uses a magnetic field and computer-generated radio waves to create detailed images of the organs and tissues within a patient, or that depicts metabolic activity of the anatomy of a patient. Many MRI machines are large, tube-shaped machines that include magnets. When a patient lies inside such an MRI machine, the magnetic field temporarily realigns water molecules in the body. Radio waves cause these aligned atoms to produce faint signals, which are used to create cross-sectional MRI images. In some embodiments, the MRI machine can also produce 3D images that can be viewed from different angles. In some embodiments, the database 136 contains a series of cross-sectional MRI images and stores the data in the sequence they are captured by the imaging device. In some embodiments, the procedure data may be the type of surgery, the 3D image of the patient's medical images, the tools required for the surgery, the process required for the surgery, including the data files for replays of the step as inputted into the CAD GUI 142 and the x, y, z coordinates of the patient's body and the tools used, the techniques used, such as the threading technique used in the surgery, the data files for the robotic movements of the process or techniques, the calculations, such as the forces required in certain steps or techniques, the materials required for certain steps or techniques, and the specialists required for the specific steps or techniques.

According to some embodiments, as discussed above, a replay may be used to generate 2D/3D representations of a patient, which may be viewed from a plurality of orientations, and can allow temporal scrolling and/or real time playback. According to some embodiments, this can allow inspection of all available data by a computerized system, such as, but not limited to, using machine learning to evaluate each acquired frame from a plurality of perspectives. Accordingly, in some embodiments, the inspection can be enabled via trained professionals, such as radiologists, surgeons, etc. In some embodiments, segments, from specific perspectives, may be clipped and used to help summarize the report's findings. In some embodiments, a replay may comprise a log of the actions taken such that the procedure could be reproduced according to the previous procedure. In some embodiments, a replay may facilitate review for training and quality assurance purposes, as discussed above.

For example, if the procedure for a disc herniation is a microdiscectomy, the procedure data may include the data for the surgical robot 102 and the workflow for the surgeon, such as making a 1 to 1½-inch incision in the midline of the low back, the back muscles (erector spinae) are lifted off the bony arch (lamina) of the spine and moved to the side. Since these back muscles run vertically, they are held to the side with a retractor during the surgery; they do not need to be cut. The surgeon may be able to enter the spine by removing a membrane over the nerve roots (ligamentum flavum). Operating glasses (loupes) or an operating microscope allow the surgeon to clearly visualize the nerve root. In some cases, a small portion of the inside facet joint is removed both to facilitate access to the nerve root and to relieve any pressure or pinching on the nerve. The surgeon may make a small opening in the bony lamina (called a laminotomy) to access the operative site. The nerve root is gently moved to the side. The surgeon uses small instruments to go under the nerve root and remove the fragments of disc material that have extruded out of the disc. The muscles are moved back into place. The surgical incision is closed, and steri-strips are placed over the incision to help hold the skin in place to heal. In some embodiments, the surgical robot 102 may perform all or a portion of the procedure or assist the surgeon in some manner during the procedure.

An example of a pathology database 138 will now be explained with reference to FIG. 8. The database 138 may contain the historical patient data of previous patients (such as each patient's ID), the indication from the radiology report, the image data, the type of image data, the annotation data, the notes data, the impression from the radiology report, the procedure performed on the patient, the procedure data, and the surgical robot data.

An example of a recommendation database 140 will now be explained with reference to FIG. 9. A recommendation database 140 may contain the probability scores generated by an analysis by an analysis module 130, in which historical patient data stored in the pathology database 138 is compared to a current patient's data to determine if a radiology report is similar and may indicate that a current patient may have a similar procedure as a previous patient had performed, if approved by a surgeon, physician, medical professional, etc. The database 140 may contain the patient's ID, the probability score, the indication from the radiology report, the image data, the type of image data, the annotation data, the notes data, the impression from the radiology report, the procedure performed on the patient, the procedure data, and the surgical robot data.

Advanced surgical systems include many different types of equipment to monitor and anesthetize the patient, assist the surgeon in performing surgical tasks, and maintain the environment of the operating room. Non-limiting examples of surgical equipment that may be used or improved by techniques described herein are provided for reference.

Vital signs monitor refers to medical diagnostic instruments and in particular to a portable, battery powered, multi-parametric, vital signs monitoring device that can be used for both ambulatory and transport applications as well as bedside monitoring. These devices can be used with an isolated data link to an interconnected portable computer allowing snapshot and trended data from the monitoring device to be printed automatically and also allowing default configuration settings to be downloaded to the monitoring device. The monitoring device is capable of use as a stand-alone unit as well as part of a bi-directional wireless communications network that includes at least one remote monitoring station. A number of vital signs monitoring devices are known that are capable of measuring multiple physiologic parameters of a patient wherein various sensor output signals are transmitted either wirelessly or by means of a wired connection to at least one remote site, such as a central monitoring station. A vital signs monitor can be integrated into the embodiments in a variety of manners, decorated

Heart rate monitor refers to the sensor(s) and/or sensor system(s) that can be applied in the context of monitoring heart rates. Embodiments are intended to measure, directly or indirectly, any physiological condition from which any relevant aspect of heart rate can be gleaned. For example, some of the embodiments measure different or overlapping physiological conditions to measure the same aspect of heart rate. Alternatively, some embodiments measure the same, different, or overlapping physiological conditions to measure different aspects of heart rate, i.e., number of beats, strength of beats, regularity of beats, beat anomalies, etc. A heart rate monitor can be integrated into the embodiments in a variety of manners.

Pulse oximeter or SpO2 Monitor refers to a plethysmograph or any instrument that measures variations in the size of an organ or body part on the basis of the amount of blood passing through or present in the part. An oximeter is a type of plethysmograph that determines the oxygen saturation of the blood. One common type of oximeter is a pulse oximeter. A pulse oximeter is a medical device that indirectly measures the oxygen saturation of a patient's blood (as opposed to measuring oxygen saturation directly through a blood sample) and changes in blood volume in the skin. A pulse oximeter may include a light sensor that is placed at a site on a patient, usually a fingertip, toe, forehead, or earlobe, or in the case of a neonate, across a foot. Light, which may be produced by a light source integrated into the pulse oximeter, containing both red and infrared wavelengths is directed onto the skin of the patient and the light that passes through the skin is detected by the sensor. The intensity of light in each wavelength is measured by the sensor over time. The graph of light intensity versus time is referred to as the photoplethysmogram (PPG) or, more commonly, simply as the “pleth”. From the waveform of the PPG, it is possible to identify the pulse rate of the patient and when each individual pulse occurs. In addition, by comparing the intensities of two wavelengths when a pulse occurs, it is possible to determine blood oxygen saturation of hemoglobin in arterial blood. This relies on the observation that highly oxygenated blood will relatively absorb more red light and less infrared light than blood with a lower oxygen saturation. A pulse oximeter can be integrated into the embodiments in a variety of manners.

End Tidal CO2 monitor or capnography monitor refers to an instrument which is used for measurement of level of carbon dioxide (referred to as end tidal carbon dioxide, ETCO2) that is released at the end of an exhaled breath. End Tidal CO2 monitor or capnography monitor is widely used in anesthesia and intensive care. ETCO2 can be calculated by plotting expiratory CO2 with time. Further, ETCO2 monitor plays a very crucial role for the measurement of applications such as Cardiopulmonary Resuscitation (CPR), Airway assessment, Procedural sedation and analgesia, pulmonary diseases such as obstructive pulmonary disease, pulmonary embolism, etc., heart failure, metabolic disorders, etc. The instrument can be configured as side stream (diverting) or mainstream (non-diverting). Diverting device transports, a portion of a patient's respired gases from the sampling site to the sensor while non-diverting device does not transport gas away. Also, measurement by the instrument is based on the absorption of infrared light by carbon dioxide, where exhaled gas passes through a sampling chamber containing an infrared light source and photodetector on both sides. Based on the amount of infrared light reaching the photodetector, the amount of carbon dioxide present in the gas can be calculated. An ETCO2 monitor or capnography monitor can be integrated into the embodiments in a variety of manners.

Blood pressure monitor refers to any instrument that measures blood pressure, particularly in arteries. Blood pressure monitors use a non-invasive technique (by external cuff application) or an invasive technique (by a cannula needle inserted in artery, used in operating theatre) for measurement, with non-invasive measurement being widely used. The non-invasive method (referred to as sphygmomanometer further) works by measurement of force exerted against arterial walls during ventricular systole (i.e., systolic blood pressure, occurs when heart beats and pushes blood through the arteries) and ventricular diastole (i.e., diastolic blood pressure, occurs when heart rests and is filling with blood) thereby measuring systole and diastole, respectively. It can be of three types automatic/digital, manual (aneroid-dial), and manual (mercury-column). The sphygmomanometer may include a bladder, a cuff, a pressure meter, a stethoscope, a valve, and a bulb. The cuff then inflates until it fits tightly around your arm, cutting off your blood flow, and then the valve opens to deflate it. It operates by inflating a cuff tightly around the arm, as the cuff reaches the systolic pressure, blood begins to flow around your artery, and creating a vibration which is detected by the meter, which records your systolic pressure. This systolic pressure is recorded. The techniques used for measurement may be: auscultatory or oscillometric. A blood pressure monitor can be integrated into the embodiments in a variety of manners.

Body temperature monitor refers to any instrument which is used for measurement of body temperature. The instrument can measure the temperature invasively or non-invasively by placement of sensor into organs such as bladder, rectum, esophagus, tympanum, esophagus, etc., and mouth, rectum, armpit, etc., respectively. The sensors are of two types: contact and non-contact. It can be measured in two forms: core temperature and peripheral temperature. Temperature measurement can be done by these sensing technologies: thermocouples, resistive temperature devices (RTDs, thermistors), infrared radiators, bimetallic devices, liquid expansion devices, molecular change-of-state, and silicon diodes. A thermometer which is a commonly used instrument for the measurement of temperature consists of a temperature sensing element (e.g., temperature sensor) and a means for converting to a numerical value. A blood temperature monitor can be integrated into the embodiments in a variety of manners.

Respiration rate or breathing rate is the rate at which breathing occurs and is measured by a number of breaths a person takes per minute. The rate is usually measured when a person is at rest and simply involves counting the number of breaths for one minute by counting how many times the chest rises. Normal respiration rates for an adult person at rest are in the range: 12 to 16 breaths per minute. A variation can be an indication of an abnormality/medical condition or a patient's demographic parameters. Hypoxia is a condition with low levels of oxygen in the cells and hypercapnia is a condition in which high levels of carbon dioxide in the bloodstream. Pulmonary disorders, asthma, anxiety, pneumonia, heart diseases, dehydration, drug overdose are some of the abnormal conditions which can bring a change to the respiration rate, thereby increasing or reducing the respiration rate from normal levels. Respiratory rate can be integrated into the embodiments in a variety of manners.

An electrocardiogram abbreviated as EKG or ECG refers to a representation of the electrical activity of the heart (graphical trace of voltage versus time) which is done by placement of electrodes on skin/body surface. The electrodes capture the electrical impulse which travels through the heart causing systole and diastole or the pumping of the heart. This impulse gives a lot of information related to the normal functioning of the heart and the production of impulses. A change may occur due to medical conditions such as arrhythmias (tachycardia where the heart rate becomes faster and bradycardia where the heart rate becomes slower), coronary heart disease, heart attacks, cardiomyopathy. The instrument used for the measurement of the electrocardiogram is called an electrocardiogramhich measures the electrical impulses by the placement of electrodes on the surface of the body and represents the ECG by a PQRST waveform. PQRST wave is read as: P wave which represents the depolarization of the left and right atrium and corresponding to atrial contraction, QRS complex indicates ventricular depolarization and represents the electrical impulse as it spreads through the ventricles; T wave indicates ventricular repolarization and follows the QRS complex. An electrocardiogram can be integrated into the embodiments in a variety of manners.

Neuromonitoring also called Intraoperative neurophysiological monitoring (abbreviated as IONM) refers to an assessment of functions and changes in the brain, brainstem, spinal cord, cranial nerves, and peripheral nerves during a surgical procedure on these organs. It includes both continuous monitoring of neural tissue as well as the localization of vital neural structures. IONM measures changes in these organs which are indicative of irreversible damage, injuries in the organs, aiming at reducing the risk of neurological deficits after operations involving the nervous system. This has also been found to be effective in localization of anatomical structures, including peripheral nerves and sensorimotor cortex, which help in guiding the surgeon during dissection. Electrophysiological modalities which are employed in neuromonitoring are an extracellular single unit and local field recordings (LFP), Somatosensory Evoked Potential (SSEP), transcranial electrical motor evoked potentials (TCeMEP), Electromyography (EMG), electroencephalography (EEG), and auditory brainstem response (ABR). The use of neurophysiological monitoring during surgical procedures requires specific anesthesia techniques to avoid interference and signal alteration due to anesthesia. Neuromonitoring can be integrated into the embodiments in a variety of manners.

Motor Evoked Potential abbreviated as MEP refers to electrical signals which are recorded from descending motor pathways or muscles following stimulation of motor pathways within the brain. MEP may be calculated by measurement of the action potential which is elicited by non-invasive stimulation of the motor cortex through the scalp. MEP is a widely used technique for intraoperative monitoring and neurophysiological testing of the motor pathways specifically during spinal procedures. The technique of monitoring for measurement of MEP can be defined based on some of the parameters like a site of stimulation (motor cortex or spinal cord), method of stimulation (electrical potential or magnetic field), and site of recording (spinal cord or peripheral mixed nerve and muscle). The target site may be stimulated by the use of electrical or magnetic means. MEP can be integrated into the embodiments in a variety of manners.

Somatosensory evoked potential abbreviated as SSEP, or SEP refers to the electrical signals which are elicited by the brain and the spinal cord in response to sensory stimulus or touch. SSEP is one of the most frequently used techniques for intraoperative neurophysiological monitoring in spinal surgeries. The method proves to be very reliable which allows for continuous monitoring during a surgical procedure. However, accuracy may be a concern at times in measurement. The sensor stimulus which is commonly given to the organs may be auditory, visual, or somatosensory SEPs and applied on the skin, peripheral nerves of the upper limb, lower limb, or scalp. The stimulation technique may be mechanical (widely used), or electrical (found to give larger and more robust responses), intraoperative spinal monitoring modality. Somatosensory evoked potential can be integrated into the embodiments in a variety of manners.

Electromyography abbreviated as EMG refers to the evaluation and recording of electrical signals or electrical activity of the skeletal muscles. Electromyography instrument or Electromyograph or Electromyogram, the instrument for the measurement of the EMG activity works on a technique used for a recording of electrical activity produced by skeletal muscles and evaluation of the functional integrity of individual nerves. The nerves which are monitored by the EMG instrument may be intracranial, spinal, or peripheral nerves. The electrodes which may be used for the acquisition of signals may be invasive and non-invasive electrodes. The technique used for measurement may be spontaneous or triggered. Spontaneous EMG refers to the recording of myoelectric signals during surgical manipulation such as compression, stretching, or pulling of nerves produces; and does not perform external stimulation. Spontaneous EMG may be recorded by the insertion of a needle electrode. Triggered EMG refers to the recording of myoelectric signals during stimulation of target site such as pedicle screw with incremental current intensities. Electromyography can be integrated into the embodiments in a variety of manners.

Electroencephalography abbreviated as EEG refers to the electrical signals in the brain. Brain cells communicate with each other through electrical impulses. EEG can be used to help detect potential problems associated with this activity. An electroencephalograph is used for the measurement of EEG activity. Electrodes ranging from 8 to 16 pairs are attached to the scalp where each pair of electrodes transmit a signal to one or more recording channels. It is one of the oldest and most commonly utilized modalities for intraoperative neurophysiological monitoring and assessing cortical perfusion and oxygenation during a variety of vascular, cardiac, and neurosurgical procedures. The waves produced by EEG are Alpha, Beta, Theta, and Delta. Electroencephalography can be integrated into the embodiments in a variety of manners.

Medical visualization systems refer to visualization systems that are used for visualization and analysis of objects (preferably three-dimensional (3D) objects). Medical visualization systems include the selection of points at surfaces, selection of a region of interest, selection of objects. Medical visualization systems may be used for applications diagnosis, treatment planning, intraoperative support, documentation, educational purpose. Medical visualization systems may consist of microscopes, endoscopes/arthroscopes/laparoscopes, fiber optics, surgical lights, high-definition monitors, operating room cameras, etc. 3D visualization software provides visual representations of scanned body parts via virtual models, offering significant depth and nuance to static two-dimensional medical images. The software facilitates improved diagnoses, narrowed surgical operation learning curves, reduced operational costs, and shortened image acquisition times. Medical visualization systems can be integrated into the embodiments in a variety of manners.

A microscope refers to an instrument that is used for viewing samples & objects that cannot be seen with an unaided eye. A microscope may have components eyepiece, objective lenses, adjustment knobs, stage, illuminator, condenser, diaphragm. A microscope works by manipulating how light enters the eye using a convex lens, where both sides of the lens are curved outwards. When light reflects off of an object being viewed under the microscope and passes through the lens, it bends towards the eye. This makes the object look bigger than it is. A microscope may be of types compound (light illuminated and the image seen with the microscope is two dimensional), dissection or stereoscope (light illuminated and image seen with the microscope is three dimensional), confocal (laser-illuminated and image seen with the microscope on a digital computer screen), Scanning Electron abbreviated as SEM (electron illuminated and image seen with the microscope in black and white), Transmission Electron Microscope abbreviated as TEM (electron illuminated and image seen with the microscope is the high magnification and high resolution). A microscope can be integrated into the embodiments in a variety of manners.

Endoscopes or arthroscopes or laparoscopes refer to minimally invasive surgical techniques where procedures are performed by performing minimal incision in the body. An Endoscope refers to an instrument to visualize, diagnose, and treat problems inside hollow organs where the instrument is inserted through natural body openings such as the mouth or anus. An endoscope may perform a procedure as follows: scope with a tiny camera attached to a long, thin tube is inserted. The doctor moves it through a body passageway or opening to see inside an organ. It can be used for diagnosis and surgery (such as for removing polyps from the colon). Arthroscope refers to an instrument to visualize, diagnose, and treat problems inside a joint by a TV camera inserted through small portals/incisions and perform procedures on cartilage, ligaments, tendons, etc. An endoscope may perform the procedure as follows: a surgeon makes a small incision in a patient's skin and inserts a pencil-sized instrument with a small lens and lighting system to magnify the target site (joint) and viewing of the interior of the joint by means of a miniature television camera and performing procedure. Endoscope refers to an instrument to visualize, diagnose, and treat problems inside soft organs like the abdomen and pelvis by a TV camera inserted through small portals/incisions and perform procedures. Endoscopes/arthroscopes/laparoscopes or minimally invasive surgery techniques can be integrated into the embodiments in a variety of manners.

Fiber optics refers to flexible, transparent fiber made by drawing glass (silica) or plastic to a diameter slightly thicker than that of a human hair. Fiber optics are arranged in bundles called optical cables and used to transmit light signals over long distances. Fiber optics are used most often as a means to transmit light between the two ends of the fiber and find wide usage in the medical field. Traditional surgery requires sizable and invasive incisions to expose internal organs and operate on affected areas and with fiber optics much smaller surgical incisions can be performed. Fiber optics contain components core, cladding, buffer coating. Fiber optics may be inserted in hypodermic needles and catheters, endoscope, operation theatres, ophthalmology, dentistry tools. Fiber optics sensors comprise a light source, optical fiber, external transducer, and photodetector. Fiber-optic sensors may be intrinsic or extrinsic. Fiber optics sensors may be categorized into four types physical, imaging, chemical, and biological. Fiber optics can be integrated into the embodiments in a variety of manners.

Surgical lights also referred to as operating light refers to an instrument that performs illumination of a local area or cavity of the patient. Surgical lights play an important role in illumination before, during, and after a medical procedure. Surgical lights may be categorized by lamp type as conventional (incandescent) and LED (light-emitting diode). Surgical lights may be categorized by mounting configuration as ceiling-mounted, wall-mounted, or floor stand. Surgical lights may be categorized by type as tungsten, quartz, and/or xenon halogens and light-emitting diodes (LEDs). Surgical lights include sterilizable handles which allow the surgeon to adjust light positions. Some important factors affecting surgical lights may be illumination, shadow management (cast shadows and contour shadows), the volume of light, heat management, fail-safe surgical lighting. Surgical lights can be integrated into the embodiments in a variety of manners.

High-definition monitors refer to a display in which a clearer picture than possible with low-definition, low-resolution screens. High-definition monitors have a higher density of pixels per inch than past standard TV screens. Resolution for high-definition monitors may be 1280×720 pixels or more. Full HD—1920×1080, Quad HD—2560×1440, 4K—3840× 2160, 8K—7680×4320 pixels. High-definition monitor may operate in progressive or interlaced scanning mode. High definition monitors used in medical applications may offer the following advantages improved visibility and allows for precise and safe surgery, rich color reproduction and provides suitable colors for each clinical discipline, better visibility, and operability with a large screen and electronic zoom, higher image quality in low light conditions, high contrast at high spatial frequencies, twice as sensitive as conventional sensors, easier determination of tissue boundaries (fat, nerves, vessels, etc.), better visualization of blood vessels and lesions. High-definition monitors can be integrated into the embodiments in a variety of manners.

Operating room cameras refer to cameras that collect images from 360 degrees, and sensors that monitor both the operating room and people in it. Operating room cameras consist of cameras that are equipped in system and perform recording to give a bird's-eye view to the surgical team. Some cameras are on devices that surgeons insert through small incisions or orifices to see what they are doing during minimally invasive surgery. Operating room cameras may perform recording for this purpose: educational purposes: example—to broadcast a live feed of a surgical demonstration to a remote audience, to collect authentic footage for edited, instructional videos on a surgical technique or procedure; to facilitate video enhanced debriefing and coaching, or to formally assess surgical skills. Operating room cameras can be integrated into the embodiments in a variety of manners.

Surgical tower refers to an instrument used for performing minimally invasive surgery or surgery which is performed by creating small incisions in the body, therefore they are also referred to as minimally invasive devices or minimally invasive access devices. The procedure of performing minimally invasive surgery may be referred to as minimally invasive procedure or minimally invasive surgery, abbreviated as MIS. MIS is a safe, less invasive, and precise surgical procedure. Some of the advantages offered by surgical towers may be small incisions, less pain, low risk of infection, short hospital stays, quick recovery time, less scarring, and reduced blood loss. Some medical procedures where surgical towers are useful and are widely used may be lung procedures, gynecological, head and neck, heart, and urological conditions. MIS may be robotic or non-robotic/endoscopic. MIS may include the following: endoscopic, laparoscopic, arthroscopic, natural orifice intraluminal, and natural orifice transluminal procedures. A surgical tower access device may be designed as an outer sleeve and an inner sleeve that telescoping or slidably engages with one another. When a telescope is used to operate on the abdomen, the procedure is called laparoscopy. Surgical towers typically include access to a variety of surgical tools, such as, for example, electrocautery, radiofrequency, lasers, sensors, etc. A surgical tower can be integrated into the embodiments in a variety of manners.

Electrocautery refers to an instrument that is used for burning a part of the body to remove or close off a part of it. Various physiological conditions or surgical procedures require the removal of body tissues and organs, a consequence of which is bleeding. In order to achieve hemostasis and for removing and sealing all blood vessels which are supplied to an organ after surgical incision an electrocautery instrument may be used. For example: after removing part of the liver for removal of tumor etc., blood vessels in the liver must be sealed individually. An electrocautery instrument may be used for sealing living tissue such as arteries, veins, lymph nodes, nerves, fats, ligaments, and other soft tissue structures. It may be used in applications surgery, tumor removal, nasal treatment, wart removal. Electrocautery may operate in modes two monopolar or bipolar. The electrocautery instrument may consist of a generator, a handpiece, and one or more electrodes. Electrocautery can be integrated into the embodiments in a variety of manners.

Radiofrequency (RF) is used in association with minimally invasive surgery devices. The radiofrequency (RF) may be used for the treatment of skin by delivering it to the skin through a minimally invasive tool (fine needles) which does not require skin excision. The RF may be used for real-time tracking of minimally invasive surgery devices such as laparoscopic instruments. The RF may provide radiofrequency ablation to a patient suffering from atrial fibrillation through smaller incisions made between the ribs. The RF may be used to perform an endoscopic surgery on the body such as the spine by delivery of RF energy. Radiofrequency can be integrated into the embodiments in a variety of manners.

Laser is used in association with minimally invasive surgery devices. The laser may be used in minimally invasive surgeries with an endoscope. The laser is attached to the distal end of the endoscope and steers the laser at high speed by producing higher incision quality than existing surgical tools and minimizing damage to surrounding tissue. Laser may be used to perform minimally invasive surgeries using an endoscope, laparoscope in the lower and upper gastrointestinal tract, eye, nose, and throat. Lasers are used in minimally invasive surgery to ablate soft tissues, such as a herniated spinal disc bulge. Laser can be integrated into the embodiments in a variety of manners.

Sensors are used in association with minimally invasive surgery devices. The sensor may be used in minimally invasive surgeries for tactile sensing of tool-tissue interaction forces. During minimally invasive surgeries field of view and workspace of tools are compromised due to the indirect access to the anatomy and lack of surgeon's hand-eye coordination. The sensors provide a tactile sensation to the surgeon by providing information of shape, stiffness, and texture of organ or tissue (different characteristics) to surgeon's hands through a sense of touch. This detection of a tumor through palpation, which exhibit a ‘tougher’ feel than healthy soft tissue, pulse felt from blood vessels, and abnormal lesions. The sensors may provide in output shape, size, pressure, softness, composition, temperature, vibration, shear, and normal forces. Sensor may be electrical or optical, consisting of capacitive, inductive, piezoelectric, piezoresistive, magnetic, and auditory. The sensors may be used in robotic, laparoscopic, palpation, biopsy, heart ablation, and valvuloplasty. Sensors can be integrated into the embodiments in a variety of manners.

Imaging systems refer to techniques or instruments which are used for the creation of images and visualization of the interior of a human body for diagnostic and treatment purposes. Imaging systems play a crucial role in every medical setting and can help in the screening of health conditions, diagnosing causes of symptoms, monitor health conditions. Imaging systems may include various imaging techniques such as X-ray, Fluoroscopy, Magnetic resonance imaging (MRI), Ultrasound, Endoscopy, Elastography, Tactile imaging, Thermography, Medical photography, and nuclear medicine e.g., Positron emission tomography (PET). Some factors which may drive the market are cost and clinical advantages of medical imaging modalities, a rising share of ageing populations, increasing prevalence of cardiovascular or lifestyle diseases, increasing demand from emerging economies. Some factors which may inhibit the market are saturation in many segments, high costs, lack of trained personnel. Imaging systems can be integrated into the embodiments in a variety of manners.

X-ray refers to a medical imaging instrument that uses X-ray radiation (i.e., X-ray range in the electromagnetic radiation spectrum) for the creation of images of the interior of the human body for diagnostic and treatment purposes. An X-ray instrument is also referred to as an X-ray generator. It is a non-invasive instrument based on different absorption of x-rays by tissues based on their radiological density (radiological density is different for bones and soft tissues). For the creation of an image by the X-ray instrument, X-rays produced by an X-ray tube are passed through a patient positioned to the detector. As the X-rays pass through the body, images appear in shades of black and white, depending on the type of tissue the X-rays pass through and their densities. Some of the applications where X-rays are used may be bone fractures, infections, calcification, tumors, arthritis, blood vessel blockages, digestive problems, heart problems. The X-ray instrument may consist of components such as an x-ray tube, operating console, collimator, grids, detector, radiographic film, etc. An X-ray can be integrated into the embodiments in a variety of manners.

Magnetic resonance imaging abbreviated as MRI refers to a medical imaging instrument that uses powerful magnets for the creation of images of the interior of the human body for diagnostic and treatment purposes. Some of the applications where MRI may be used may be brain/spinal cord anomalies, tumors in the body, breast cancer screening, joint injuries, uterine/pelvic pain detection, heart problems. For the creation of the image by an MRI instrument, magnetic resonance is produced by powerful magnets which produce a strong magnetic field that forces protons in the body to align with that field. When a radiofrequency current is then pulsed through the patient, the protons are stimulated, and spin out of equilibrium, straining against the pull of the magnetic field. Turning off the radiofrequency field allows detection of energy released by realignment of protons with the magnetic field by MRI sensors. The time taken by the protons for realignment with the magnetic field, and energy release is dependent on environmental factors and the chemical nature of the molecules. MRI may more widely suit for imaging of non-bony parts or soft tissues of the body. MRI may be less harmful as it does not use damaging ionizing radiation as in the X-ray instrument. MRI instrument may consist of magnets, gradients, radiofrequency system, computer control system. Some areas where imaging by MRI should be prohibited may be people with implants. MRI can be integrated into the embodiments in a variety of manners.

Computed tomography imaging abbreviated as CT refers to a medical imaging instrument that uses an X-ray radiation (i.e., X-ray range in the electromagnetic radiation spectrum) for the creation of cross-sectional images of the interior of the human body for diagnostic and treatment purposes. CT refers to a computerized x-ray imaging procedure in which a narrow beam of x-rays is aimed at a patient and quickly rotated around the body, producing signals that are processed by the machine's computer to generate cross-sectional images—or “slices”—of the body The CT instrument produces cross-sectional images of the body. Computed tomography instrument is different from an X-ray instrument as it creates 3-dimensional cross-sectional images of the body while X-ray creates 2-dimensional images of the body; the 3-dimensional cross-sectional images are created by taking images from different angles, which is done by taking a series of tomographic images from different angles. The different taken images are collected by a computer and digitally stacked to form a three-dimensional image of the patient. For creation of images by the CT instrument, a CT scanner uses a motorized x-ray source that rotates around the circular opening of a donut-shaped structure called a gantry while the x-ray tube rotates around the patient shooting narrow beams of x-rays through the body. Some of the applications where CT may be used may be blood clots, bone fractures, including subtle fractures not visible on X-ray, organ injuries. CT can be integrated into the embodiments in a variety of manners.

Stereotactic navigation systems refer to an instrument that uses patient imaging (e.g., CT, MRI) to guide surgeons in the placement of specialized surgical instruments and implants before and during a procedure. The patient images are taken to guide the physician before or during the medical procedure. The stereotactic navigation system includes a camera having infrared sensors to determine the location of the tip of the probe being used in the surgical procedure. This information is sent in real-time so that the surgeons have a clear image of the precise location of where they are working in the body. Stereotactic navigation systems may be framed (attachment of a frame to patient's head using screws or pins) or frameless (do not require the placement of a frame on the patient's anatomy). Stereotactic navigation systems may be used for diagnostic biopsies, tumor resection, bone preparation/implant placement, placement of electrodes, otolaryngologic, or neurosurgical procedures. Stereotactic navigation systems can be integrated into the embodiments in a variety of manners.

Ultrasound imaging also referred to as sonography or ultrasonography refers to a medical imaging instrument that uses ultrasound or sound waves (also referred to as acoustic waves) for the creation of cross-sectional images of the interior of the human body for diagnostic and treatment purposes. Ultrasound in the instrument may be produced by a piezoelectric transducer which produces sound waves and sends them into the body. The sound waves which are reflected are converted into electrical signals which are sent to an ultrasound scanner. Ultrasound instruments may be used for diagnostic and functional imaging. Ultrasound instruments may be used for therapeutic or interventional procedures. Some of the applications where ultrasound may be used are diagnosis/treatment/guidance during medical procedures e.g., biopsies, internal organs such as liver/kidneys/pancreas, fetal monitoring, etc., in soft tissues, muscles, blood vessels, tendons, joints. Ultrasound may be used for internal (transducer is placed in organs e.g., vagina) and external (transducer is placed on chest for heart monitoring or abdomen for the fetus). An ultrasound machine may consist of a monitor, keyboard, processor, data storage, probe, and transducer. Ultrasound can be integrated into the embodiments in a variety of manners.

Anesthesiology machine refers to a machine that is used to generate and mix medical gases like oxygen or air and anesthetic agents to induce and maintain anesthesia in patients. Anesthesiology machines deliver oxygen and anesthetic gas to the patient as well as filter out expiratory carbon dioxide. Anesthesia machine may perform following functions provides O2, accurately mix anesthetic gases and vapors, enable patient ventilation, and minimize anesthesia related risks to patients and staff. Anesthesia machine may consist of the following essential components a source of oxygen (O2), O2 flowmeter, vaporizer (anesthetics include isoflurane, halothane, enflurane, desflurane, sevoflurane, and methoxyflurane), patient breathing circuit (tubing, connectors, and valves), scavenging system (removes any excess anesthetics gases). Anesthesia machine may be divided into three parts the high-pressure system, the intermediate pressure system, and the low-pressure system. The process of anesthesia starts with oxygen flow from pipeline or cylinder through the flowmeter, O2 flows through the vaporizer and picks up the anesthetic vapors, the O2-anesthetic mix then flows through the breathing circuit and into the patient's lungs, usually by spontaneous ventilation or normal respiration. The O2-anesthetic mix then flows through the breathing circuit and into the patient's lungs, usually by spontaneous ventilation or normal respiration. An anesthesiology machine can be integrated into the embodiments in a variety of manners.

Surgical bed is a bed equipped with mechanisms that can elevate or lower the entire bed platform, flex, or extend individual components of the platform, or raise or lower the head or the feet of the patient independently. Surgical bed may be an operation bed, cardiac bed, amputation Bed, fracture bed. Some essential components of a surgical bed may be bed sheet, woolen blanket, bath towel, bed block. Surgical beds can also be referred to as a postoperative bed, refers to a special type of bed made for the patient who is coming from the operation theatre or from another procedure that requires anesthesia. The surgical bed is designed in a manner that makes it easier to transfer an unconscious or weak patient from a stretcher/wheelchair to the bed. The surgical bed should protect bed linen from vomiting, bleeding, drainage, and discharges, provide warmth and comfort to the patient to prevent shock, provide necessary position, which is suitable for operation, protect patient from being chilled, prepared to meet any emergency. Surgical bed can be integrated into the embodiments in a variety of manners.

Disposable air warmer (also referred to as bair) refers to a convective temperature management system used in a hospital or surgery center to maintain a patient's core body temperature. The instrument consists of a reusable warming unit and a single-use disposable warming blankets for use during surgery and may also be used before and after surgery. The air warmer uses convective warming consisting of two components a warming unit and a disposable blanket. The air warmer filter air and then force warm air through disposable blankets which cover the patient. The blanket may be designed to use pressure points on the patient's body to prevent heat from reaching areas at risk for pressure sores or burns. The blanket may also include drain holes where fluid passes through the surface of the blanket to linen underneath which will reduce the risk of skin softening and reduce the risk of unintended cooling because of heat loss from evaporation. Disposable air warmer can be integrated into the embodiments in a variety of manners.

Sequential compression device abbreviated as SVD refers to an instrument that is used to help prevent blood clots in the deep veins of legs. The sequential compression device use cuffs around the legs that fill with air and squeeze your legs. This increases blood flow through the veins of your legs and helps prevent blood clots. A deep vein thrombosis (DVT) is a blood clot that forms in a vein deep inside the body. Some of the risks of using a DVT may be discomfort, warmth, or sweating beneath the cuff, skin breakdown, nerve damage, pressure injury. Sequential compression device can be integrated into the embodiments in a variety of manners.

Jackson frame refers to a frame or table which is designed for use in spine surgeries and may be used in a variety of spinal procedures in supine, prone, lateral positions in a safe manner. Two peculiar features of the Jackson table are no central table support and its ability to rotate the table through 180 degrees. The Jackson table is supported at both ends keeping the whole of the table free. This allows the visualization of trunk and major parts of extremities as well. The Jackson frame allows the patient to be slid from the cart onto the table in the supine position with appropriate padding placed. The patient is then strapped securely on the table. The Jackson frame can be integrated into the embodiments in a variety of manners.

Bed position controller refers to an instrument for controlling the position of the patient bed. Positioning a patient in bed is important for maintaining alignment and for preventing bedsores (pressure ulcers), foot drop, and contractures. Proper positioning is also vital for providing comfort for patients who are bedridden or have decreased mobility related to a medical condition or treatment. When positioning a patient in bed, supportive devices such as pillows, rolls, and blankets, along with repositioning, can aid in providing comfort and safety. The patient may be in the following positions in a bed supine position, prone position, lateral position, sims position, fowler's position, semi-Fowler's position, orthopedic or tripod position, Trendelenburg position. Bed position controller can be integrated into the embodiments in a variety of manners.

Operating room environmental controls refers to control or maintenance of the environment in an operation theatre where procedures are performed to minimize the risk of airborne infection and provide a conducive environment for everyone in the operation theatre-surgeon, anesthesiologist, nurses & patient). Some factors which may contribute to poor quality in the environment of the operating room are temperature, ventilation, and humidity and they can lead to profound effects on the health of people in the operating room and work productivity. As an example: surgeons prefer a cool, dry climate since they work in bright, hot lights; anesthesia personnel prefer a warmer, less breezy climate; patient condition demands a relatively warm, humid, and quiet environment. Operating room environmental controls may control the environment by taking care of the following factors environmental humidity, infection, odor control. Humidity control may be done by controlling the temperature of anesthesia gases; Infection can be controlled by the use of filters to purify the air. Operating room environmental controls can be integrated into the embodiments in a variety of manners.

Heating, ventilation, and air conditioning (abbreviated as HVAC) refers to a system for regulating environment of indoor settings by moving air between indoor and outdoor areas, along with heating and cooling. HVAC may use a different combination of systems, machines, and technologies to improve comfort. HVAC may be necessary to maintain the environment of an operating room. HVAC for an operating room may be a traditional operating room (which may have a large diffuser array directly above the operating table) or a hybrid operating room (which may have monitors and imaging equipment that consume valuable ceiling space and complicate the design process). HVAC may consist of three main units heating unit (it may be a furnace or a boiler), a ventilation unit (it may be natural or forced), and an air conditioning unit (which may remove existing heat). HVAC may be made of components as air return, filter, exhaust outlets, ducts, electrical elements, outdoor unit, compressor, coils, and blower. The HVAC system may use central heating and AC systems that use a single blower to circulate air via internal ducts. Heating, ventilation, and air conditioning can be integrated into the embodiments in a variety of manners.

Air purification refers to a system for removing contaminants from the air in a room to improve indoor air quality. Air purification may be important in an operating room as surgical site infection may be a reason for high mortality and morbidity. The air purification system may deliver clean, filtered, contaminant-free air over the operating room table with diffuser, airflow, etc., to remove all infectious particles down and away from the patient. Air purification system may be air curtain, multi-diffuser array, or single large diffuser (based on laminar diffuser flow) or High-Efficiency Particulate Air filter. High-Efficiency Particulate Air filter referred to as HEPA filter protects from infection and contamination by a filter which is mounted at the terminal of the duct. HEPA filter may be mounted on the ceiling and deliver clean, filtered air in a flow to the room that provides a sweeping effect that pushes contaminants out via the return grilles that are usually mounted on the lower wall. Air purification can be integrated into the embodiments in a variety of manners.

Orthopedic tools also referred to as orthopedic instruments used for treatment and prevention of deformities and injuries of musculoskeletal system or skeleton, articulations, and locomotive system (i.e., set formed by skeleton, muscles attached to it and part of nervous system which controls the muscles). Major percentage of orthopedic tools are made of plastic. Orthopedic tools may be divided into the following specialties hand and wrist, foot and ankle, shoulder and elbow, arthroscopy, hip, and knee. Orthopedic tool may be fixation tools, relieving tools, corrective tools, compression-distraction tools. Fixation tool refers to a tool designed to restrict movements partially or completely in a joint, e.g., hinged splints (for preserving a certain range of movement in a joint), rigid splints. Relieving tool refers to a tool designed to relieve pressure on an ailing part by transferring support to healthy parts of an extremity, e.g., Thomas splint and the Voskoboinikova apparatus. Corrective tool refers to a tool designed to gradually correct a deformity, e.g., corsets, splints, orthopedic footwear, and insoles and other devices to correct abnormal positions of the foot. Compression-distraction tool refers to a tool designed to correct acquired or congenital deformities of the extremities, e.g., curvature, shortening, and pseudarthrosis such as Gudushauri. Fixation tools may be internal fixation tools (e.g., screws, plates) or external fixation tools (radius, tibia fracture fixation). Orthopedic tools may be bone-holding forceps, drill bits, nail pins, hammer staple, etc. Orthopedic tools can be integrated into the embodiments in a variety of manners.

Drill refers to a tool for making holes in bones for insertion of implants like nails, plates, screws, and wires. The drill tool functions by drilling cylindrical tunnels into bone. Drill may be used in orthopedics for performing medical procedures. Use of drill on bones may have some risks harm caused to bone, muscle, nerves, and venous tissues are wrapped by surrounding tissue, the drill does not stop immediately. Drills vary widely in speed, power, and size. Drill may be powered as electrical, pneumatic, or battery. Drills generally may work on speed below 1000 rpm in orthopedic. Temperature control of drill is an important aspect in the functioning of drill and is dependent on parameters rotation speed, torque, orthotropic site, sharpness of the cutting edges, irrigation, cooling systems. The drill may consist of components physical drill, cord power, electronically motorized bone drill, rotating bone shearing incision work unit. Drill can be integrated into the embodiments in a variety of manners.

Scalpel refers to a tool for slicing or cutting or osteotomy of bone during orthopedic procedure. The scalpel may be designed to provide clean cuts through osseous structures with minimal loss of viable bone while sparing adjacent elastic soft tissues largely unaffected while performing a slicing procedure. This is suited for spine applications where bone must be cut adjacent to the dura and neural structures. The scalpel does not rotate and performs cutting by an ultrasonically oscillating or forward/backward moving metal tip. Scalpel may prevent injuries caused by a drill in a spinal surgery such as complications such as nerve thermal injury, grasping soft tissue, tearing dura mater, and a mechanical injury may occur during drilling. Scalpel can be integrated into the embodiments in a variety of manners.

Stitches (also referred to as sutures) refers to a sterile, surgical thread used to repair cuts or lacerations and are used to close incisions or hold body tissues together after a surgery or an injury. Stitches may involve the use of a needle along with an attached thread. Stitches may be of type absorbable (the stitches automatically break down harmlessly in the body over time without intervention) and non-absorbable (the stitches do not automatically break down over time and must be manually removed if not left indefinitely). Stitches may be of type based on material monofilament, multifilament, and barb. Stitches may be classified based on size. Stitches may be of type based on material synthetic and natural. Stitches may be of type based on coating coated and un-coated. Stitches can be integrated into the embodiments in a variety of manners.

Stapler refers to a tool for fragment fixation when inter-fragmental screw fixation is not easy. When there is vast damage and bone is broken into fragments then staples can be used between these fragments for internal fixation and bone reconstruction. For example, they may be used around joints as in ankle and foot surgeries, in cases of soft tissue damage, to attach tendons or ligaments to the bone for reconstruction surgery. Stapler may be made of surgical grade stainless steel or titanium, and they are thicker, stronger, and larger. The stapler can be integrated into the embodiments in a variety of manners.

Equipment refers to a set of articles, tools, or objects which help to implement or achieve an operation or activity. A medical equipment refers to an article, instrument, apparatus, or machine used for diagnosis, prevention, or treatment of a medical condition or disease or detection, measurement, restoration, correction, or modification of structure/function of the body for some health purpose. The medical equipment may perform functions invasively or non-invasively. The medical equipment may consist of components sensor/transducer, signal conditioner, display, data storage unit, etc. The medical equipment works by taking a signal from a measurand/patient, a transducer for converting one form of energy to electrical energy, signal conditioner such as an amplifier, filters, etc., to convert the output from the transducer into an electrical value, display to provide a visual representation of measured parameter or quantity, a storage system to store data which can be used for future reference. A medical equipment may perform any function of diagnosis or provide therapy, for example, the equipment delivers air/breaths into the lungs and moves it out of the lungs and out of lungs, to a patient who is physically unable to breathe, or breaths insufficiently. A medical equipment can be integrated into the embodiments in a variety of manners.

Ventilator (also referred to as a respirator) refers to an instrument that provides a patient with oxygen when they are unable to breathe on their own. The ventilator is required when a person is not able to breathe on their own. The ventilator may perform a function of pushing air into the lungs and allows it to come back out, gently like lungs when they are working. Ventilator functions by delivery of positive pressure to force air into your lungs, while usual breathing uses negative pressure by the opening of the mouth, and air flows in. The machine uses positive pressure to force air into your lungs. A ventilator may be required during surgery or after surgery. A ventilator may be required in case of respiratory failure due to acute respiratory distress syndrome, head injury, asthma, lung diseases, drug overdose, neonatal respiratory distress syndrome, pneumonia, sepsis, spinal cord injury, cardiac arrest, etc., or during surgery. The ventilator may be used with a face mask (non-invasive ventilation, where the ventilation is required for a shorter duration of time) or with a breathing tube also referred to as an endotracheal tube (invasive ventilation, where the ventilation is required for a longer duration of time). A ventilator use may have some risks such as infections, fluid build-up, muscle weakness, lung damage, etc. A ventilator may be operated in modes ACV, SIMV, PCV, PSV, PCIRV, APRV, etc. A ventilator may have components gas delivery system, power source, control system, safety feature, gas filter, monitor. A ventilator can be integrated into the embodiments in a variety of manners.

Continuous positive airway pressure abbreviated as CPAP refers to an instrument which used for the treatment of sleep apnea disorder in a patient. Sleep apnea refers to a disorder in which breathing repeatedly stops and starts while a patient is sleeping, often because throat/airways briefly collapse or something temporarily blocks them and may lead to serious health problems, such as high blood pressure and heart trouble. Continuous positive airway pressure instrument helps the patient with sleep apnea to breathe more easily during sleep by sending a steady flow of oxygen into the nose and mouth during sleep, which keeps the airways open and helps to breathe normally. The CPAP machine may work by a compressor/motor which generates a continuous stream of pressurized air which travels through an air filter into a flexible tube. The tube delivers purified air into a mask sealed around the nose/mouth of the patient. The airstream from the instrument pushes against any blockages, opening the airways so lungs receive plenty of oxygen, and breathing does not stop as nothing obstructs oxygen. This helps the patient to not wake up to resume breathing. CPAP may have a nasal pillow mask, nasal mask, or full mask. CPAP instrument may consist of components a motor, a cushioned mask, a tube that connects the motor to the mask, a headgear frame, adjustable straps. The essential components may be a motor, a cushioned mask, a tube that connects the motor to the mask. Continuous positive airway pressure instruments can be integrated into the embodiments in a variety of manners.

Consumables refer to necessary supplies for health systems to provide care within a hospital or surgical environment. Consumables may include gloves, gowns, masks, syringes, needles, sutures, staples, tubing, catheters, and adhesives for wound dressing, in addition to other tools needed by doctors and nurses to provide care. Depending on the device mechanical testing may be carried out in tensile, compression or flexure, in dynamic or fatigue, or impact or with the application of torsion. Consumables may be disposable (are timesaving, no risk of healthcare-associated infections, cost-efficient) or sterilizable (cross-contamination, risk of surgical site infections, sterilization). Consumables can be integrated into the embodiments in a variety of manners.

Robotic systems may include systems that provide intelligent services and information by interacting with their environment, including human beings, via the use of various sensors, actuators, and human interfaces. These are employed for automating processes in a wide range of applications, ranging from industrial (manufacturing), domestic, medical, service, military, entertainment, space, etc. The adoption of robotic systems provides several benefits, including efficiency and speed improvements, lower costs, and higher accuracy. Performing medical procedures with the assistance of robotic technology may use medical robotic systems. The medical robotic system market can be segmented by product type into Surgical Robotic Systems, Rehabilitative Robotic Systems, Non-invasive Radiosurgery Robots, Hospital & Pharmacy Robotic Systems. Robotic technologies have offered valuable enhancements to medical or surgical processes through improved precision, stability, and dexterity. Robots in medicine help by relieving medical personnel from routine tasks, and by making medical procedures safer and less costly for patients. They can also perform accurate surgery in tiny places and transport dangerous substances. Robotic surgeries are performed using tele-manipulators, which use the surgeon's actions on one side to control the “effector” on the other side. A medical robotic system ensures precision and may be used for remotely controlled, minimally invasive procedures. The systems comprise computer-controlled electromechanical devices that work in response to controls manipulated by the surgeons. Robotic systems can be integrated into the embodiments in a variety of manners.

An Electronic Health Record (EHR) refers to a digital record of a patient's health information, which may be collected and stored systematically over time. It is an all-inclusive patient record and could include demographics, medical history, history of present illness (HPI), progress notes, problems, medications, vital signs, immunizations, laboratory data, and radiology reports. A computer software is used to capture, store, and share patient data in a structured way. The EHR may be created and managed by authorized providers and can make health information instantly accessible to authorized providers across practices and health organizations-such as laboratories, specialists, medical imaging facilities, pharmacies, emergency facilities, etc. The timely availability of EHR data can enable healthcare providers to make more accurate decisions and provide better care to the patients by effective diagnosis and reduced medical errors. Besides providing opportunities to enhance patient care, it may also be used to facilitate clinical research by combining all patients' demographics into a large pool. For example, the EHR data can support a wide range of epidemiological research on the natural history of disease, drug utilization, and safety, as well as health services research. The EHR can be integrated into the embodiments in a variety of manners.

Equipment tracking systems, such as RFID, refers to a system that tags an instrument with an electronic tag and tracks it using the tag. Typically, this could involve a centralized platform that provides details such as location, owner, contract, and maintenance history for all equipment in real-time. A variety of techniques can be used to track physical assets, including Radio-frequency Identification (RFID), Global Positioning System (GPS), Bluetooth Low Energy (BLE), barcodes, Near-Field Communication (NFC), Wi-Fi, etc. The equipment tracking system comprises the hardware components, such as RFID tags, GPS trackers, barcodes, and QR codes. The hardware component is placed on the asset, and it communicates with the software (directly or via a scanner), providing it with data about the asset's location and properties. An equipment tracking system uses electromagnetic fields to transmit data from an RFID tag to a reader. Reading of RFID tags may be done by portable or mounted RFID readers. RFID may be very short for low frequency or high frequency for ultra-high frequency. Managing and locating important assets is a key challenge for tracking medical equipment. Time spent searching for critical equipment can lead to expensive delays or downtime, missed deadlines and customer commitments, and wasted labor. The problem has been solved by the use of barcode labels or using manual serial numbers and spreadsheets; however, these require manual labor. The RFID tag may be passive (smaller and less expensive, read ranges are shorter, have no power of their own, and are powered by the radio frequency energy transmitted from RFID readers/antennas) or active (larger and more expensive, read ranges are longer, have a built-in power source and transmitter of their own). Equipment tracking systems may offer advantages, no line of sight required, read Multiple RFID objects at once, scan at a distance, and flexibility. Equipment tracking systems, RFID can be integrated into the embodiments in a variety of manners.

Quantum computing refers to any computational device or method which utilizes properties of quantum states defined by quantum mechanics such as superposition, entanglement, etc. to perform computations. These devices utilize qubits which are the quantum equivalent to bits in a classical computing system, comprised of at least two quantum states or probable outcomes. These outcomes, combined with a coefficient representing the probability of each outcome, describes the possible states, or bits of data, which can be represented by the qubits according to the principle of quantum superposition. These states may be manipulated which may shift the probability of each outcome or additionally add additional possible outcomes to perform a calculation, the final state of which can be measured to achieve the result.

Quantum computing provides significant benefits in the areas of encryption and the simulation of natural systems. Encryption is aided by the uncertain nature of quantum computing in that data is represented by an indeterminate state of probable outcomes, therefore making decryption virtually impossible. The simulation of natural systems, such as chemical and biological interactions, benefit from the fact that nature of quantum computing is the same as the systems being simulated. In medical fields, quantum computing shows the greatest promise for drug discovery and simulating the interaction of drugs with biologic systems, however the same technology might be used to predict the interaction of a biologic system with an implanted device, preventing rejection of an implant by a patient's body, long term function of an implant, and potentially the reaction of a patient to a surgical procedure during a simulation before a procedure or actively during a procedure.

Techniques operating according to the principles described herein may be implemented in any suitable manner. Included in the discussion above are a series of flow charts showing the steps and acts of various processes for generation of a radiology report using patient data for prior patients, based on a comparison of data. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of techniques described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and may result from compiling of other code into machine language code or intermediate code that is interpreted by a framework or virtual machine for execution.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, as a thread of a process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include functions, routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a software package or software program application. These functional facilities may, in alternative embodiments, be adapted to interact with other, unrelated functional facilities and/or processes, to implement a software package or software program application.

Some examples of modules have been described herein, which may be implemented as one or more functional facilities for carrying out one or more tasks. It should be appreciated, though, that the modules and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

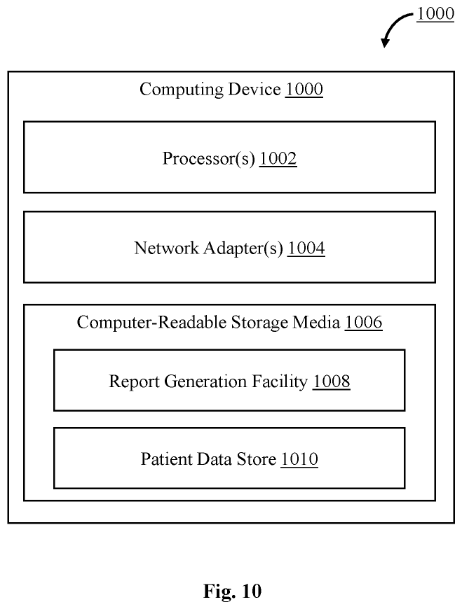

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1006 of FIG. 10 described below (i.e., as a portion of a computing device 1000) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 10 illustrates one exemplary implementation of a computing device in the form of a computing device 700 that may be used in a system implementing techniques described herein, although others are possible. It should be appreciated that FIG. 10 is intended neither to be a depiction of necessary components for a computing device to perform radiology report generation in accordance with the principles described herein, nor a comprehensive depiction.

Computing device 1000 may comprise at least one processor 1002, a network adapter 1004, and computer-readable storage media 1006. Computing device 1000 may be, for example, a desktop or laptop personal computer, a personal digital assistant (PDA), a smart mobile phone, a server, a wireless access point or other networking element, or any other suitable computing device. Network adapter 1004 may be any suitable hardware and/or software to enable the computing device 1000 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 1006 may be adapted to store data to be processed and/or instructions to be executed by processor 1002. Processor 1002 enables processing of data and execution of instructions. The data and instructions may be stored on the computer-readable storage media 1006.

The data and instructions stored on computer-readable storage media 1006 may comprise computer-executable instructions implementing techniques which operate according to the principles described herein. In the example of FIG. 10, computer-readable storage media 1006 stores computer-executable instructions implementing various facilities and storing various information as described above. Computer-readable storage media 1006 may store a report generation facility 1008 that may carry out one or more of the techniques described herein. The media 1006 may also store a patient data store 1010 that may store one or more of the types of information described above, including the data stored in any of the illustrative databases described above.

While not illustrated in FIG. 10, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

1. A method comprising:
comparing, by a device, data regarding a patient to prior patient data for one or more prior patients, the data regarding the patient comprising information regarding a medical imaging of the patient and the prior patient data for each of the one or more prior patients comprising information regarding a prior medical imaging of a prior patient, the comparing comprises generating, via execution of an image recognition algorithm by the device, for each of the prior patient data regarding the one or more prior patients, a measure of a match between the data regarding the patient and prior patient data for a prior patient;
determining, by the device, based at least in part on the measures of match between the data regarding the patient and the prior patient data for each prior patient of the one or more prior patients, there is a match between the data regarding the patient and first prior patient data for a first prior patient of the one or more prior patients;
generating, by the device, based on the determination of the match, a radiology report, the radiology report comprising the medical imaging of the patient and a recommendation for the patient, wherein the recommendation includes a recommended procedure to treat a condition of the patient and procedure data indicating surgical robot movements for the recommended procedure, wherein the generating comprises generating the radiology report based at least in part on data of the first prior patient data for which the match was determined; and
outputting, by the device, for display within a user interface (UI), the generated radiology report.

2. The method of claim 1, wherein the medical imaging of the patient comprises information related to at least one of medical image data, sensor data and notes data related to a dictation of a clinician regarding the medical image data and/or the patient.

3. The method of claim 1, wherein comparing the data regarding the patient to the prior patient data for the one or more prior patients comprises, for a prior patient:
determining one or more first items of information included in the data regarding the patient;
determining one or more second items of information included in the prior patient data for the prior patient; and
generating the measure of the match between the data regarding the patient and the prior patient data for the prior patient based on comparing the one or more first items of information and the one or more second items of information.

4. The method of claim 3, wherein:
the one or more first items of information comprise an indication and the medical image data for the medical imaging for the patient;
the one or more second items of information comprise notes of a clinician regarding the prior medical imaging of the prior patient; and
comparing the one or more first items of information and the one or more second items of information comprises comparing the indication and the medical image data for the patient to the notes of the clinician regarding the prior medical imaging of the prior patient.

5. The method of claim 4, wherein the generating the radiology report based at least part on data of the first prior patient data for which the match was determined comprises populating the radiology report with information regarding an impression of a clinician for the prior patient and/or a prior procedure performed for the prior patient.

6. The method of claim 1, wherein comparing the data regarding the patient to the prior patient data for the one or more prior patients comprises analyzing the data regarding the patient and the prior patient data for the one or more prior patients using at least one trained model, the measure of the match being an output of the at least one trained model.

7. The method of claim 1, wherein generating the radiology report based at least part on data of the first prior patient data for which the match was determined comprises copying text from the prior report into the radiology report, wherein copying text from the prior report into the radiology report comprises editing the text to reflect at least one value from the data regarding the patient.

8. The method of claim 1, wherein:
generating the radiology report comprises populating a report template;
generating the radiology report comprises populating the report template based on at least the medical image data, sensor data, and content related to notes data relating to dictation of a clinician regarding the patient; and
generating the radiology report based at least part on data of the first prior patient data for which the match was determined comprises, for one or more parts of the report template not populated based on the data regarding the patient, populating the one or more parts based on content from one or more corresponding parts of the first prior patient data.

9. The method of claim 1, wherein determining whether there is a match between the data regarding the patient and the first prior patient data for a first prior patient of one or more prior patients comprises identifying, from among one or more potential matches in the one or more prior patients, a prior patient associated with a highest measure of a match.

10. At least one computer-readable storage medium having encoded thereon executable instructions that, when executed by at least one processor, cause the at least one processor to carry out a method comprising:
comparing data regarding a patient to prior patient data for one or more prior patients, the data regarding the patient comprising information regarding a medical imaging of the patient and the prior patient data for each of the one or more prior patients comprising information regarding a prior medical imaging of a prior patient, the comparing comprises generating, via execution of an image recognition algorithm, for each of the prior patient data regarding the one or more prior patients, a measure of a match between the data regarding the patient and the prior patient data for a prior patient;
determining, based at least in part on the measures of match between the data regarding the patient and the prior patient data for each prior patient of the one or more prior patients, there is a match between the data regarding the patient and first prior patient data for a first prior patient of the one or more prior patients;
generating, based on the determination of the match, a radiology report, the radiology report comprising the medical imaging of the patient and a recommendation for the patient, wherein the recommendation includes a recommended procedure to treat a condition of the patient and procedure data indicating surgical robot movements for the recommended procedure, wherein the generating comprises generating the radiology report based at least in part on data of the first prior patient data for which the match was determined; and
outputting, for display within a user interface (UI), the generated radiology report.

11. The at least one computer-readable storage medium of claim 10, wherein comparing the data regarding the patient to the prior patient data for the one or more prior patients comprises, for a prior patient:
determining one or more first items of information included in the data regarding the patient;
determining one or more second items of information included in the prior patient data for the prior patient; and
generating the measure of the match between the data regarding the patient and the prior patient data for the prior patient based on comparing the one or more first items of information and the one or more second items of information.

12. The at least one computer-readable storage medium of claim 11, wherein:
the one or more first items of information comprise an indication and the medical image data for the medical imaging for the patient;
the one or more second items of information comprise notes of a clinician regarding the medical imaging of the prior patient; and
comparing the one or more first items of information and the one or more second items of information comprises comparing the indication and the medical image data for the patient to the notes of the clinician regarding the medical imaging of the prior patient.

13. The at least one computer-readable storage medium of claim 12, wherein generating the radiology report based at least in part on data of the first prior patient data for which the match was determined comprises populating the radiology report with information regarding an impression of a clinician for the prior patient and/or a procedure performed for the prior patient.

14. The at least one computer-readable storage medium of claim 10, wherein generating the radiology report based at least part on data of the first prior patient data for which the match was determined comprises copying text from the prior report into the radiology report.

15. The at least one computer-readable storage medium of claim 14, wherein copying text from the prior report into the radiology report comprises editing the text to reflect at least one value from the data regarding the patient.

16. An apparatus comprising:
at least one processor; and
at least one storage medium having encoded thereon executable instructions that, when executed by the at least one processor, cause the at least one processor to carry out a method comprising:
comparing data regarding a patient to prior patient data for one or more prior patients, the data regarding the patient comprising information regarding a medical imaging of the patient and the prior patient data for each of the one or more prior patients comprising information regarding a medical imaging of a prior patient, the comparing comprises generating, via execution of an image recognition algorithm, for each of the prior patient data regarding the one or more prior patients, a measure of a match between the data regarding the patient and the prior patient data for a prior patient;
determining, based at least in part on the measures of match between the data regarding the patient and the prior patient data for each prior patient of the one or more prior patients, there is a match between the data regarding the patient and first prior patient data for a first prior patient of the one or more prior patients;
generating, based on the determination of the match, a radiology report, the radiology report comprising the medical imaging of the patient and a recommendation for the patient, wherein the recommendation includes a recommended procedure to treat a condition of the patient and procedure data indicating surgical robot movements for the recommended procedure, wherein the generating comprises generating the radiology report based at least in part on data of the first prior patient data for which the match was determined; and
outputting, for display within a user interface (UI), the generated radiology report.

17. The apparatus of claim 16, wherein comparing the data regarding the patient to the prior patient data for the one or more prior patients comprises, for a prior patient:
determining one or more first items of information included in the data regarding the patient;
determining one or more second items of information included in the prior patient data for the prior patient; and
generating the measure of the match between the data regarding the patient and the prior patient data for the prior patient based on comparing the one or more first items of information and the one or more second items of information.

18. The apparatus of claim 17, wherein:
the one or more first items of information comprise an indication and the medical image data for the medical imaging for the patient;
the one or more second items of information comprise notes of a clinician regarding the medical imaging of the prior patient; and
comparing the one or more first items of information and the one or more second items of information comprises comparing the indication and the medical image data for the patient to the notes of the clinician regarding the medical imaging of the prior patient.

19. The apparatus of claim 18, wherein generating the radiology report based at least part on data of the first prior patient data for which the match was determined comprises populating the radiology report with information regarding an impression of a clinician for the prior patient and/or a procedure performed for the prior patient.

20. The apparatus of claim 16, wherein comparing the data regarding the patient to the prior patient data for the one or more prior patients comprises analyzing the data regarding the patient and the prior patient data for the one or more prior patients using at least one trained model, the measure of the match being an output of the at least one trained model.
